(19) United States
(12) Patent Application Publication
(10) Pub. No.: US 2011/0288615 A1
(43) Pub. Date: Nov. 24, 2011

(54) Implantable Therapeutic Systems Including Neurostimulation Circuits, Devices, Systems and Methods

(75) Inventors: Scott Armstrong, Bee Cave, TX (US); Lawrence J. Cauller, Plano, TX (US); Ravi Jain, Austin, TX (US); David Michael Pierce, Plano, TX (US); Guenter H. Lehman, Sachse, TX (US)

(73) Assignees: The Board of Regents, The University of Texas System, Austin, TX (US); MicroTransponder, Inc., Austin, TX (US)

(21) Appl. No.: 13/095,542

(22) Filed: Apr. 27, 2011

Related U.S. Application Data
(63) Continuation-in-part of application No. 12/323,854, filed on Nov. 26, 2008.
Application No. 12/323,854 is a continuation-in-part of application No. 12/485,040, filed on Jun. 15, 2009.
Application No. 12/485,040 is a continuation-in-part of application No. 12/611,105, filed on Nov. 2, 2009.
(60) Provisional application No. 61/328,619, filed on Apr. 27, 2010.
(60) Provisional application No. 61/329,058, filed on Apr. 28, 2010.
(60) Provisional application No. 61/332,164, filed on May 6, 2010.
(60) Provisional application No. 61/449,603, filed on Mar. 4, 2011.
(60) Provisional application No. 60/990,278, filed on Nov. 26, 2007.
(60) Provisional application No. 61/077,648, filed on Jul. 2, 2008.
(60) Provisional application No. 61/078,954, filed on Jul. 8, 2008.
(60) Provisional application No. 61/086,116, filed on Aug. 4, 2008.
(60) Provisional application No. 61/149,387, filed on Feb. 3, 2009.

(51) Int. Cl. A61N 1/375; A61N 1/372

(52) U.S. Cl. 607/59; 607/2; 607/60

(57) ABSTRACT

A neurostimulation array comprising a first implantable neurostimulator storing a first identification code in a non-volatile memory and responding to communications including said first identification code, a second implantable neurostimulator storing a second identification code in a non-volatile memory and responding to communications including said second identification code, and a polymer connector attached to said first implantable neurostimulator and said second implantable neurostimulator, thereby forming a neurostimulation array.

### CROSS-REFERENCE TO RELATED APPLICATIONS

This application claims the benefit of U.S. Provisional Patent Application No. 61/328,619, filed Apr. 27, 2010, U.S. Provisional Patent Application No. 61/329,058, filed Apr. 28, 2010, U.S. Provisional Patent Application No. 61/332,164, filed May 6, 2010, and U.S. Provisional Patent Application No. 61/449,603, filed Mar. 4, 2011. This application is also a continuation-in part of: U.S. patent application Ser. No. 12/323,854, filed Nov. 26, 2008, which claims the benefit of U.S. Provisional Patent Application No. 60/990,278, filed Nov. 26, 2007; U.S. patent application Ser. No. 12/485,040, filed Jun. 15, 2009, which claims the benefit of U.S. Provisional Patent Application No. 61/077,648, filed Jul. 2, 2008, U.S. Provisional Patent Application No. 61/078,954, filed Jul. 8, 2008, U.S. Provisional Patent Application No. 61/086,116, filed Aug. 4, 2008, and U.S. Provisional Patent Application No. 61/149,387, filed Feb. 3, 2009; and U.S. patent application Ser. No. 12/611,105, filed Nov. 2, 2009. All of these applications are incorporated herein by reference as if reproduced in their entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Implantable devices can provide a variety of therapeutic functions, including neural stimulation. Neural stimulation has proven to be an effective therapy and therapeutic component. A variety of nerves can be stimulated with a variety of therapeutic effects. For example, stimulation of the vagus nerve is effective in preventing seizures in patients suffering from severe epilepsy. Stimulation of peripheral nerves is used to generate paresthesia in patients suffering from chronic pain, such as carpal tunnel syndrome. Some headaches are effectively treated by stimulating the occipital nerve.

Effective neural stimulation may require placing a neuro stimulation device in close proximity to the nerves being stimulated. Typically, this requires the neurostimulation device to be implanted under the skin, to place the electrodes of an electric-neuro stimulation device within a millimeter of the nerve tissue so that the electric fields generated between the electrodes interact with the nerve. The interaction may evoke an action potential, energize tissue so that an action potential is more likely, and hyperpolarize the cell so that it is harder to be activated or prevent it from evoking an action potential when it normally would.

The neuro stimulator operation may require power sufficient to provide stimulation energy to the nerve. This may be accomplished by using a percutaneous lead to the neuro stimulator, but the percutaneous lead may be at risk of causing infection. The neuro stimulator may include a battery, but the presence of a battery typically requires further surgical procedures to replace the battery even when the battery is rechargeable, as recharging is possible for a limited number of recharge cycles. Transmitted energy has also been used to power implanted neuro stimulators.

Finally, a system using a fully implanted device may be more acceptable or desirable when the implanted device has the ability to communicate with external devices, such as to confirm proper operation.

### SUMMARY

For purposes of summarizing the disclosure, certain aspects, advantages, and novel features of the disclosure have been described herein. It is to be understood that not necessarily all such advantages may be achieved in accordance with any particular embodiment of the disclosure. Thus, the disclosure may be embodied or carried out in a manner that achieves or optimizes one advantage or group of advantages as taught herein without necessarily achieving other advantages as may be taught or suggested herein.

In an embodiment the disclosure includes a neurostimulation array comprising a first implantable neurostimulator storing a first identification code in a non-volatile memory and responding to communications including said first identification code, a second implantable neurostimulator storing a second identification code in a non-volatile memory and responding to communications including said second identification code, and a polymer connector attached to said first implantable neurostimulator and said second implantable neurostimulator, thereby forming a neurostimulation array.

In another embodiment the disclosure includes an implantable neurostimulation array system comprising an array of neurostimulators, wherein each neurostimulator is represented by an individual identification code, and an external controller in communication with the neurostimulators, wherein a communication from the external controller to one of the neurostimulators comprises the individual identification code representing the one of the neurostimulators, wherein the external controller supplies power modulated with the communication to the array of neurostimulators, and wherein the neurostimulators communicate with the external controller by modulating an antenna load.

In another embodiment the disclosure includes an implantable voltage controlled capacitive discharge neuro stimulation system comprising an external controller, and an implantable neuro stimulator in communication with the external controller, wherein the implantable neurostimulator provides voltage controlled capacitive discharge stimulation energy.

In another embodiment the disclosure includes an implantable voltage controlled capacitive discharge neurostimulator comprising a power source, and a plurality of electrodes coupled to the power source, wherein the power source provides voltage controlled capacitive discharge stimulation energy to the electrodes.

In another embodiment, the disclosure includes a neurostimulation device comprising a housing that has a substantially rectangular shape including a first side and a second side on opposite sides of the substantially rectangular shape, a first electrode located on the first side, and a second electrode located on the second side.

In another embodiment the disclosure includes a neuro stimulation system comprising an implantable neuro stimulation device identified by an identification code, and an external device in communication with the implantable neuro stimulation device, wherein the external device receives the identification code from the implantable neurostimulation device, assigns a local identification code to the implantable neuro stimulation device for use in future communication, and transmits the local identification code to the implantable neuro stimulation device, and wherein the local identification code is stored in memory in the implantable neurostimulation device.

In another embodiment, the disclosure includes a neuro stimulation system comprising an implantable neurostimulation device comprising housing, and an imaging system for detecting the implantable neurostimulation device, wherein the housing is substantially radio-opaque to the imaging system from a first direction and substantially radio-transparent to said imaging system from a second direction.

In another embodiment, the disclosure includes a neuro stimulation system comprising an implantable neuro stimulation device including an internal inductive coil, and an external control device configured to communicate inductively with the implantable neuro stimulation device, wherein the implantable neuro stimulation device communicates with the external control device by changing the inductance of the internal inductive coil.

In another embodiment, the disclosure includes a neurostimulation device comprising a housing comprising a metallic electrode, wherein the housing does not cover a portion of the metallic electrode, and a non-conductive coating that covers at least part of the portion of the metallic electrode.

In another embodiment, the disclosure includes a neurostimulation device comprising a rectangular shaped housing that has a first side and a second side, a first electrode on the first side, and a second electrode on the second side.

In another embodiment the disclosure includes a neurostimulation device comprising a housing having a first face and a second face, a plurality of tri-state electrodes on the first face, and a plurality of tri-state electrodes on the second face.

In another embodiment the disclosure includes an implantable device with a hermetic seal comprising a first alumina shell piece having a first bonding surface covered with a gold-tin epoxy, a second alumina shell piece having a second bonding surface covered with a gold metallization, wherein the first alumina shell piece is bonded to the second alumina shell piece such that the first bonding surface is aligned with the second bonding surface and the gold-tin epoxy and the gold metallization form a hermetic seal.

These and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 depicts an implantable system.

FIG. 2 is a functional block diagram depicting an embodiment of an implantable device.

FIG. 3 is a functional block diagram depicting an embodiment of an external controller.

FIG. 4 is a functional block diagram depicting an embodiment of a clinical programming device.

FIG. 5 is a functional block diagram depicting an embodiment of internal power management systems.

FIG. 6 is a functional block diagram depicting an embodiment of an internal power transfer system.

FIG. 7 is a functional block diagram depicting an embodiment of an internal communication system.

FIG. 8 depicts an embodiment of a near-field power transfer system.

FIG. 9 depicts embodiments of a plurality of neurostimulators.

FIG. 10 depicts an embodiment of a pre-assembled implantable neurostimulation device.

FIG. 11 depicts the pre-assembled implantable neurostimulation device in FIG. 10 from a reverse perspective.

FIG. 12 depicts an embodiment of an assembled implantable neurostimulation device.

FIG. 13 depicts another embodiment of a pre-assembled implantable neurostimulation device.

FIG. 14 depicts the pre-assembled implantable neurostimulation device in FIG. 13 from a reverse perspective.

FIG. 15 depicts an embodiment of a pre-cut assembled implantable neuro stimulation device.

FIG. 16 depicts an embodiment of an assembled and cut implantable neuro stimulation device.

FIG. 17 depicts embodiments of a plurality of assembled neuro stimulation arrays.

FIG. 18 depicts an embodiment of an external controller.

FIG. 19 is a block diagram depicting an embodiment of an external controller circuitry.

FIG. 20 depicts an embodiment of timing of signals associated with the external controller circuitry of FIG. 19.

FIG. 21 depicts an embodiment of a plurality of transmitter loops associated with the external controller circuitry of FIG. 19.

FIG. 22 depicts an embodiment of a plurality of receiver loops associated with the external controller circuitry of FIG. 19.

FIG. 23 is a block diagram depicting an integrated circuit of an implantable device.

FIG. 24 is an embodiment of a block diagram depicting a transmission/receiving system of an external controller.

FIG. 25 is a series of graphs depicting embodiments of communication signals.

FIG. 26 is a functional block diagram depicting an embodiment of an external controller system.

FIG. 27 is a functional block diagram depicting another embodiment of the external controller system.

FIG. 28 depicts an embodiment of the external controller.

FIG. 29 depicts an embodiment of a deployed array of implantable devices.

FIG. 30 depicts an embodiment of an implantable device with remote electrodes.

FIG. 31 depicts an embodiment of an external controller with remote power coils.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents. The present application describes several embodiments, and none of the statements below should be taken as limiting the claims generally.

Where block diagrams have been used to illustrate the embodiments, it should be recognized that the physical location where described functions are performed are not necessarily represented by the blocks. Part of a function may be performed in one location while another part of the same function is performed at a distinct location. Multiple functions may be performed at the same location. In a functional block diagram, a single line may represent a connection, in general, or a communicable connection, particularly in the presence of a double line, which may represent a power connection. In either case, a connection may be tangible, as in a wire, or radiated, as in near-field communication. An arrow may typically represent the direction of communication or power although should not be taken as limiting the direction of connected flow.

Many of the functional systems described are represented in both implantable devices and the external controller. For example, there may be a communication system in the implantable device and a communication system in the external controller. Systems of the implantable device will be called “internal” and systems of the external controller will be called “external.” In this context, these terms will only refer to this distinction. For example, an internal control system is deployed within a body and an external control system is situated outside the body. Likewise, an internal coil system that is part of an implantable device is defined as being deployable within a body but is not necessarily internal with respect to the implantable device.

In the description of electrical energy provided between neuro stimulation electrodes, the energy may be described as a voltage between the electrodes, an electrical pulse, or pulse stream having a peak voltage, duration, and frequency, as currents through tissue, as capacitive coupling, and/or as charge and ion densities. Those skilled in the art will recognize the interchangeable nature of these descriptions.

The implantable system as described below may include one or more of the described aspects, taken individually and in combination. In particular, the implantable system may include a wide variety of implantable devices. For purposes of illustration, the implantable system may be described as a low-power neuro stimulation system where the implantable device is a low-power neuro stimulation device.

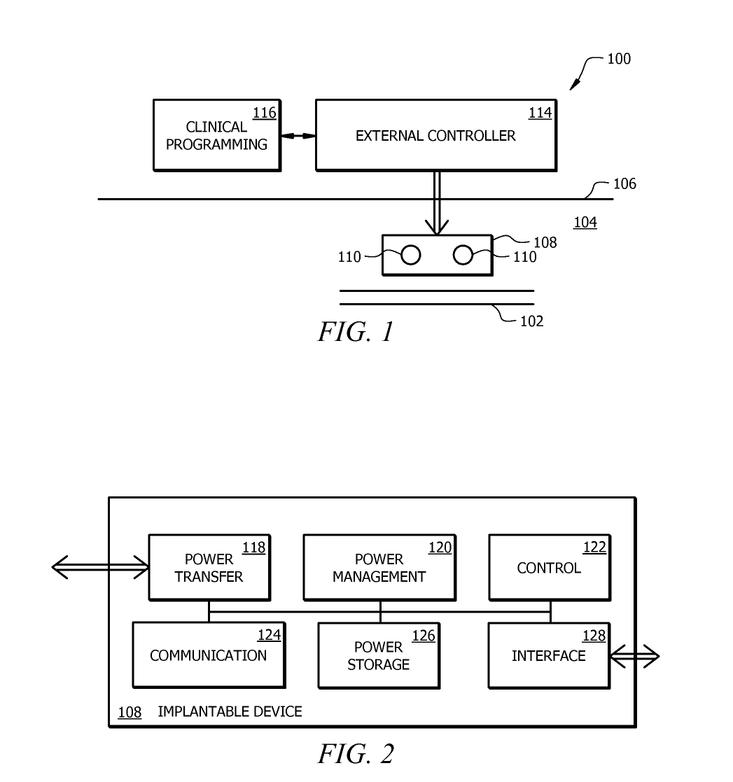

With reference to FIG. 1, an implantable system 100 is depicted. The implantable system 100 may include an implantable device 108 configured to be implanted in a living tissue layer 104 of a human being or other animal, for example, near a nerve 102. The implantable device 108 may be a stimulation device and include stimulation electrodes 110. The implantable device 108 may be in communication with an external controller 114 typically through a tissue layer 104 such as skin 106.

The external controller 114 may communicate power and data to the implantable device 108. The external controller 114 may be programmed by a clinical programming device or system 116, for example to set stimulation parameters for individual implantable devices 108 including voltage and pulse frequency.

The clinical programming device 116, such as a laptop computer, may receive and record patient data and instructions, typically provided by a clinician. The external controller 114 may collect data from the implantable device 108. The external controller 114 may communicate collected or generated data to the clinical programming device 116. The clinical programming device 116 may record and process the received data and may present the data to a clinician using a visual interface, such as a liquid crystal display (LCD) screen.

With reference to FIG. 2, a functional block diagram depicts an implantable device 108. The implantable device 108 may be a neuro stimulator, a drug-delivery device, a sensor, a therapeutic device, a component of a multi-device system, or any device suitable for implantation.

Functionally, the implantable device 108 may include several internal functional systems to enable the implantable device 108 to perform its intended tasks. Typically, the implantable device 108 includes an internal power transfer system 118 to transfer power into the implantable device 108. The implantable device 108 may include internal power management system 120 to manage the transferred power. The implantable device 108 may include internal communication system 124 to communicate with other devices, such as the external controller 114 (shown in FIG. 1). The implantable device 108 may also include internal control system 122 to control the functions of the implantable device 108. The implantable device 108 may include an internal power storage component 126, such as a batter or a capacitor. Additionally, the implantable device 108 may include an internal output delivery system or interface 128 for delivery of therapeutics such as electrostimulation, neuromodulation, drugs, or other deliverable therapeutics or for the delivery of energy to other implantable devices.

The described functions may be performed by integrated circuits, discrete components, and/or software. In accordance with an embodiment, an implantable device 108 may have an input current rating from about zero to about five milliamps, an input voltage rating from about zero to about twenty volts, a radio frequency (RF) input rating from about zero to about three milliwatts, an operating temperature from about zero to about 45 degrees Celsius (° C.), a storage temperature from about −20 to about +70° C., or combinations thereof. The implantable device 108 may also provide stimulation pulses from about zero to about 130 Hertz (Hz), from about zero to about 12 volts, or both.

The implantable device 108 may include the internal power transfer system 118, where power may be internally provided from within the implantable device 108, as with a battery. Power may be provided percutaneously. For instance, the implantable device 108 may be connected to wires that pass through the skin to make a direct connection to an external power source. Power may be provided through tissue, with the implantable device 108 connected to wires that make a direct connection to a power source that may also be implanted. Power may be generated or received, for example by harvesting local energy, by near-field coupling, far-field coupling, acoustic energy, pressure energy, light, heat, motion, or any other suitable energy source. Power may be received by an antenna. For instance, in one embodiment, the implantable device 108 may be implanted in skin tissue and may be powered inductively, e.g., using inductive coupling with an external power source outside the skin tissue. In some scenarios, power may be converted using piezoelectrics, coils, photovoltaic systems, or other suitable energy conversion devices.

The implantable device 108 may include the internal power management system 120 to manage power. When energy is being received, the implantable device 108 may include a tuning system to tune the circuits to a resonant frequency equal to the transmitted frequency or some other appropriate frequency. The internal power management system 120 may include systems to provide over-voltage protection, either by clamping the voltage or by detuning the system to reduce power transfer efficiency. The internal power management system 120 may include a rectifier system. The internal power management system 120 may also include a voltage multiplier, such as a switched capacitor array. The implantable device 108 may include the internal delivery control system 122, such as a switch matrix. The implantable device 108 may include an internal power storage component 126, for example, a capacitor.

The implantable device 108 may include the internal interface 128. The internal interface 128 may typically enable performance functions including the implantable device's intended therapeutic functions. In an implantable device 108 that provides electrical neural stimulation, the internal interface 128 may provide an electric stimulation pulse or a series of stimulation pulses at the electrodes 110 (see FIG. 1). For instance, the electric stimulation pulses may correspond to voltage controlled capacitive discharge stimulations. In an electric neuro stimulation implantable device, the stimulation pulses provided at the electrodes 110 may be in stimulation waveforms such as a capacitor discharge, constant current, constant voltage, rectangular, linear increase, linear decrease, exponential increase, exponential decrease, Gaussian sinusoidal, or any other suitable electric pulse waveform or series of waveforms that sufficiently stimulate the nerve 102. For instance, the electric neuro stimulation implantable device may be or may comprise a voltage controlled capacitive discharge (VCCD) neurostimulator.

In an implantable device 108 that includes sensing capability, the internal interface 128 may enable the sensing function, for example for measuring temperature, pressure, magnetic field, or any other measurable condition, detecting the presence of chemicals, molecules, proteins or any other detectable substance, or detecting an electrical signal such as a nerve activation impulse. In an implantable device 108 that delivers pharmaceutical or other therapeutic substances, the internal interface 128 may include a drug-delivery system, such as for releasing substances in response to conditions, instructions, or timing. In a mechanical stimulation device, the internal interface 128 may perform a mechanical function such as vibration. The internal interface 128 may also perform communication functions and/or provide power for another implantable device.

The implantable device 108 may include the internal communication system 124. The internal communication system 124 may receive communications from an external device such as an external controller 114 or from internal devices such as other implantable devices. The communications may include information regarding timing, such as a clock signal. The communications may include instructions, commands, or other suitable control data. The communications may also include parameters, for example stimulation parameters such as peak voltage, current, duration, and/or frequency. The communication may include data, firmware updates, and/or other programming data.

The internal communication system 124 may transmit communications to an external device such as an external controller 114 or to internal devices such as other implantable devices. The communications may be assigned or may include identification data, such as local identification codes associated with the implantable devices. The communications may also include an acknowledgment signal, device status data such as data regarding received power, therapeutic data, relayed data, and/or any other suitable data.

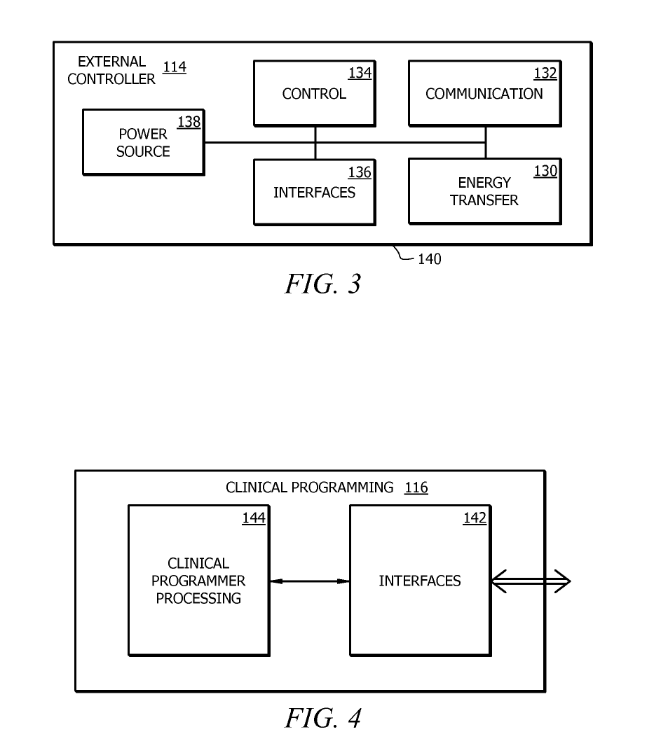

With reference to FIG. 3, a functional block diagram depicts an external controller 114. The external controller 114 may include external communication systems 132 that provide communications to and from the implantable device 108. The external controller 114 may include energy transfer systems 130 that transfer energy to the implantable device 108. In some embodiments, for example using a near-field energy transmission, the energy transfer may be modulated to communicate with the implantable device 108 while transferring energy to the implantable device 108, or may be of a different frequency or energy form.

The external controller 114 may also include an external control system 134 that control the functions of the external controller 114, for example a microprocessor, memory, and appropriate programming. The external controller 114 may include external power supply or source systems 138 that connect to a power supply, for example power sources such as alternate current (AC) or direct current (DC) power, batteries, rechargeable batteries, computer interface, or any other appropriate power source. The external controller 114 may include implantable device location systems, such as coupled load demodulation location, magnetic sensing, Hall-effect sensing, ultrasound, or any other suitable location technique.

The external controller 114 may be encased with an external housing 140 configured to permit the energy transfer system 130 to be placed next to or near the skin 106 (see FIG. 1), for example above the implantable device 108 when implanted. The external housing 140 may include a cuff, patch, accessory, article of clothing, or any other suitable arrangement. The external housing 140 may be substantially rectangular and comprise a first side (e.g., a top side) and a second side (e.g., bottom side), which may be aligned in parallel. The external controller 114 may also include an imaging system or external interfaces 136, such as a LCD display, keys, buttons, dials, knobs, switches, and/or circuits to connect the external controller 114 to other devices, including a universal serial bus (USB) connection, a wireless, network connection, or any other appropriate communication system. The external housing 140 of the external controller 114 may also include shielding to prevent radiated magnetic and electric fields from interfering with other electrical equipment, or from interfering with the external controller 114 components. The external housing 140 may be substantially radio-opaque to the imaging system or external interfaces 136 from a first direction, and may be substantially radio-transparent to the imaging system or external interfaces 136 from a second direction. For example, the external housing 140 may be substantially radio-transparent to the imaging system 136 along the axis of component placement to allow device identification and device failure analysis but may be substantially radio-opaque along the axis perpendicular (e.g., cross section axis) of the component placement.

The external control systems 134 may be provided with programmable systems, including software, to establish the identity of implantable devices 108 and recognize the identity of the implantable devices 108. The programmable systems may permit functional parameters to be set, stored, and transmitted to the implantable devices 108. The programmable systems may permit data collection. The programmable systems may also permit the upgrade of firmware used by an implantable device 108 or by the external controller 114.

With reference to FIG. 4, a functional block diagram depicts an embodiment of a clinical programming device or system 116. The clinical programming system 116 may typically be a personal computer, such as a laptop or any suitable digital device. The clinical programming system 116 may include interfaces 142 designed to allow a physician, clinician, operator, or patient to program the external controller 114. The interfaces 142 may include human-machine interfaces, such as an imaging system, graphical user interfaces, keyboards, pointing interfaces and/or other suitable human-machine interfaces. The interfaces 142 may include machine-machine interfaces between the clinical programming system 116 and the external controller 114. The machine-machine interfaces may include wired or wireless communication systems.

The clinical programming system 116 may include a clinical programmer processing unit 144, typically including a microprocessor, memory, and data storage. The clinical programmer processing unit 144 may send and receive data from the external controller 114 via the interfaces 142. The clinical programming system 116 may provide implantable identification data to the external controller 114, collect data from the external controller 114, upgrade the software of the external controller 114, and/or provide software for the upgrade of an implantable device 108.

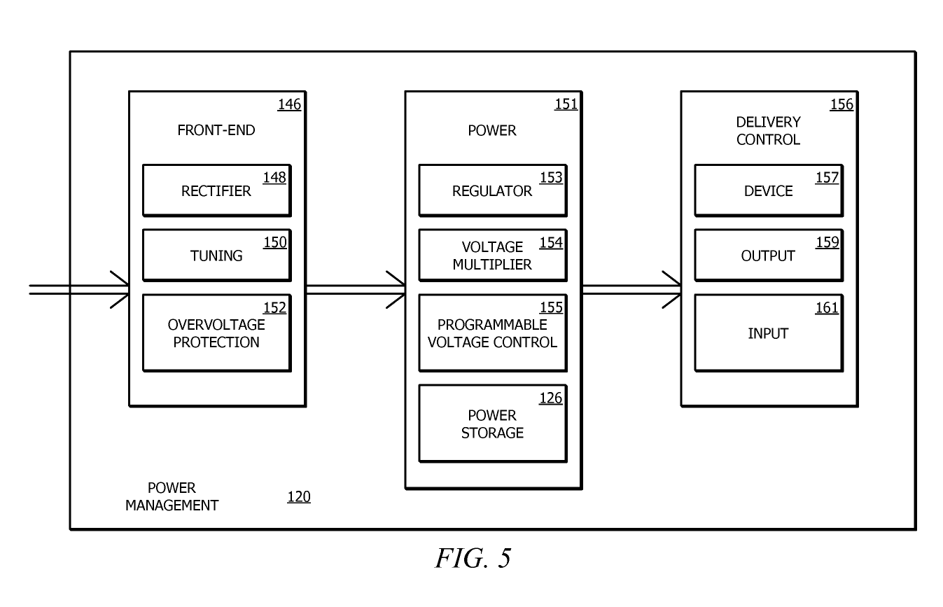

With reference to FIG. 5, a functional block diagram depicts the internal power management system 120 of an implantable device 108. The internal power management system 120 may include internal front-end systems 146, internal power systems 151, and delivery control systems 156. The internal front-end system 146 may receive, convert, and process incoming power. The internal front-end system 146 may include a rectifier 148 when the power source has alternating polarity, a tuning system 150 to tune a circuit's frequency to enhance reception, and an over-voltage protection system 152 that clamps or limits the voltage or detunes the reception to prevent damage when an excess of power is received.

The internal power systems 151 may condition power for consumption. For example, a regulator 153 in the internal power systems 151 may regulate voltages to an appropriate level for operating digital circuits. A voltage multiplier 154 may be used in the internal power systems 151 to increase the voltage levels, typically by switching capacitances or using diodes. A programmable voltage control 155 may be implemented to provide increments of voltage, for example, any voltage from about zero to about 12 volts in about 0.25-volt steps. Power may be stored in a power storage device 126, for example a capacitor in the internal power systems 151.

The delivery control system 156 may include device systems 157, output systems 159, and input systems 161. The device systems 157 may provide power to digital circuits, communication systems, memory systems or any other systems in the implantable device 108 that require power. The output systems 159 may include waveform shaping, timing controls, or a switch matrix used to direct the power to an appropriate electrode, circuit or other connection. For example, four tri-state stimulation electrodes 110 may be provided on the outer surface of an implantable device 108. The output systems 159 may determine the voltage polarity of the electrodes 110, directing the currents between them, and otherwise optimizing electrical neural stimulation. The input systems 161 may include sensing systems.

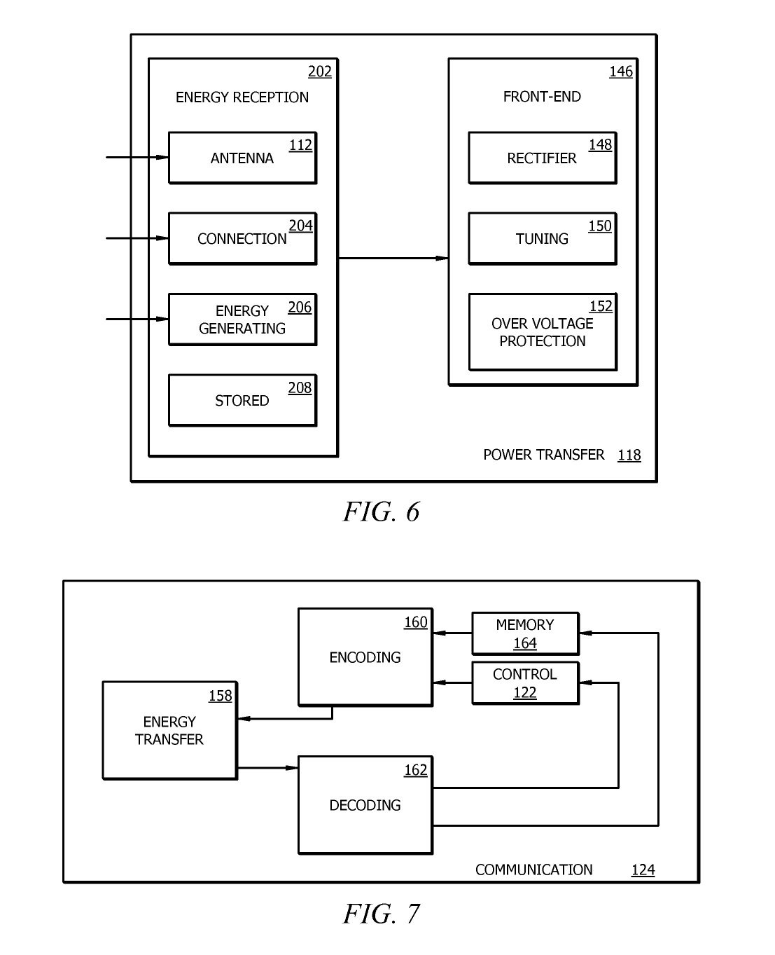

With reference to FIG. 6, a block diagram depicts functions of an internal power transfer system 118. The internal power transfer system 118 may include energy reception systems 202 and front-end systems 146. The energy reception system 202 may include an antenna system 112 for reception of radiated energy such as near-field, optical, or acoustic energy. The energy reception system 202 may include direct connection systems 204 for wired, optical fiber or otherwise direct connection to an energy source. The energy reception system 202 may also include energy generating systems 206, such as energy harvesting systems. The energy reception system 202 may include stored energy systems 208, such as battery and/or capacitor systems. The front-end systems 146 may include a rectifier 148, a tuning unit 150, and an over-voltage protection unit 152, which may be substantially the same as the corresponding components in FIG. 5.

With reference to FIG. 7, a block diagram depicts an internal communication system 124 of an implantable device 108. The internal communication system 124 may include an energy transfer circuit 158 for transmitting and receiving communication signals. An encoding system 160 may be used in the internal communication system 124 to encode data for transmission. The encoding system 160 may encode stored information in a memory 164 or may encode data generated by the internal control system 122 of the implantable device 108. The internal communication system 124 may also include the internal control system 122 described above. A decoding system or circuits 162 may also be used to decode data encoded in signals received by the energy transfer circuit or system 158. The decoded data may be stored in the memory 164 or may be provided to the internal control system 122, typically to control the operation of the implantable device 108. The internal control system 122 may process the received data to provide control of the other circuits. The internal control system 122 may process data produced by the other circuits, and may process parameters and identification data.

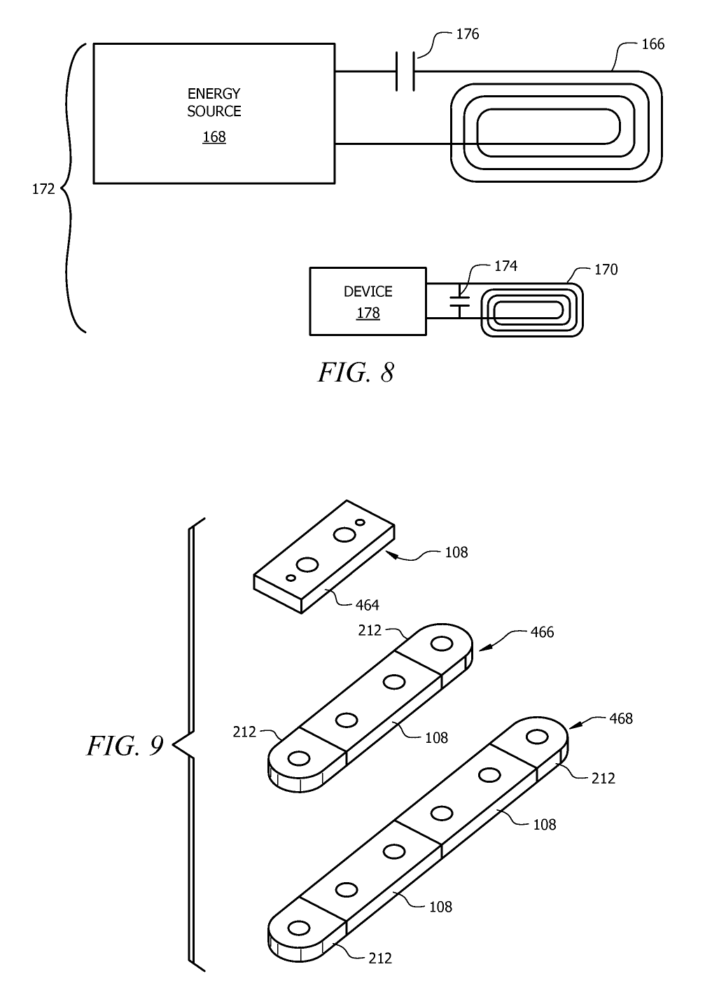

With reference to FIG. 8, a near-field power transfer system 172 is depicted. The near-field power transfer system 172 may include an internal antenna 170, such as a flat spiral coil, in combination with an internal tuning capacitor 174, and a device 178 that may correspond to the implantable device 108. The near-field power transfer system 172 may also include an external antenna 166, such as a flat spiral coil, in combination with an external tuning capacitor 176 and a RF energy source 168. The transfer of power is affected by the number of turns in each coil and the area enclosed by each coil. Communication between the coils is affected by the ratio of the diameter of the coils. In accordance with an embodiment, flat spiral coils are used for both the internal antenna 170 and the external antenna 166.

Although the general arrangement of a flat inductive coil is a spiral, the actual shape of the antenna may be circular, rectangular, square, oval, triangular, or any other suitable shape. Coils may be arranged so that they have a common axis. The planes of a flat coil may be arranged in parallel. In accordance with an embodiment, multiple coil layers may be arranged with a common axis, in parallel planes with the direction of windings remaining the same, and may be separated by an insulating material. Although the center of the coils may be open, this area may be filled with a ferromagnetic material to increase the efficiency and coupling.

In some embodiments, more than one coil may be arranged to compensate for rotation of the implantable device 108. The rotational compensation coil arrangements may compensate for specific kinds of rotation. Two coils may be arranged so that their planes are perpendicular or at an angle that compensates for rotation along the plane intersection. Three coils may be arranged mutually perpendicular to compensate for spherical rotation. Three coils may be arranged with their planes forming an equilateral triangle, to improve compensation along a single axis. The actual arrangement of coils may be varied depending on the intended position and operation of the coils. A single coil could be used to receive power across multiple planes using a center tap.

A rotational compensation arrangement may include the implantable coil on a hinged platform that can tilt in multiple directions to self-align with the maximum magnetic flux. Additionally, the transmitted RF field can be shaped by shifting the phases of the RF signal in multiple external coils such that the flux lines are maximized for any rotation of the implant.

The internal antenna 170 and the external coil 166 may be separated by a medium, such as tissue 104, and otherwise surrounded by mediums that affect the effectiveness of the near-field power transfer from the external antenna 166 and the internal antenna 170. The effectiveness of the near-field power transfer may also depend on the separation and alignment between the internal antenna 170 and the external antenna 166. The effectiveness of the near-field power transfer depends on many factors, including the resonance of the antennas 170 and 166, the magnetic field produced by the external antenna 166 and the magnetic flux density at the internal antenna 170. Additional internal antenna 170 may be used to increase the power harvested from magnetic field. The relative area of the antennas 166 and 170 may also influence the effectiveness of energy transfer.

FIG. 9 depicts a plurality of neurostimulators, in accordance with various embodiments. For instance, a neurostimulation unit that corresponds to the implantable device 108 may have a ceramic housing 464. A neuro stimulation unit or the implantable device 108 may be connected to suture hole connectors or ends 212 to form a 1×1 neurostimulation array 466. A 1×2 neuro stimulation array 468 may include two neuro stimulation units or implantable devices 108. Neuro stimulation arrays including three or more neurostimulation units or implantable devices 108 are contemplated. The number and configuration of neuro stimulation units or implantable devices 108 used in array will typically depend on the therapy, target nerve or other appropriate factors.

A typical device housing for electronic devices is formed of ceramic. Electronic packages are typically manufactured by placing components from the top down onto a circuit substrate within the device housing. Typical ceramic housings for electronic devices are hermetically sealed by laser welding, ultrasonically welding, resistance welding, or brazing a metal lid onto a ceramic and substantially hollow base that contains the circuitry and components for the device. X-ray visibility through the device housing and onto the components from the axis of component placement allows device identification and device failure analysis. A device housing including a metal lid may eliminate the ability to differentiate the device housing from the components contained within that device housing. A housing with a ceramic base and lid will generally not be substantially radio-opaque along the axis of component placement, thus allowing post-manufacturing analysis. Additionally, metallic housings or lids absorb and shield more electromagnetic energy, decreasing the efficiency or power transfer into the device.

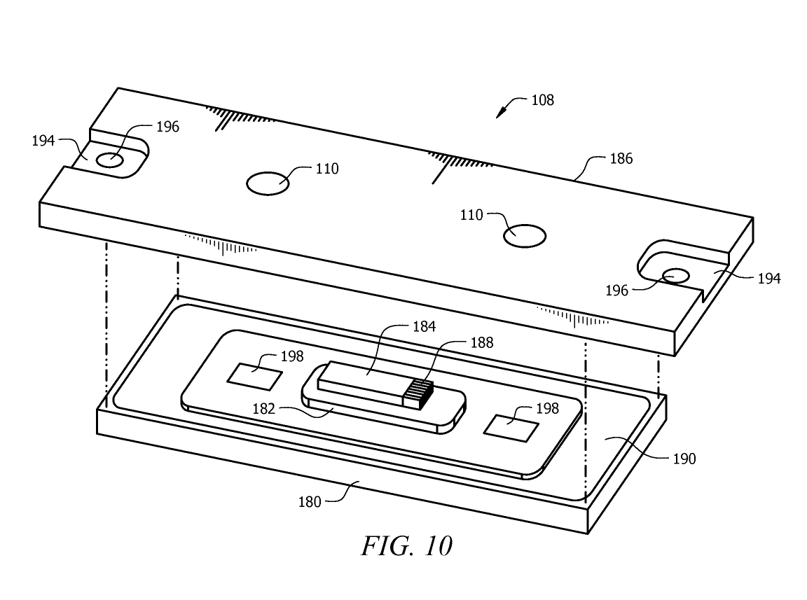

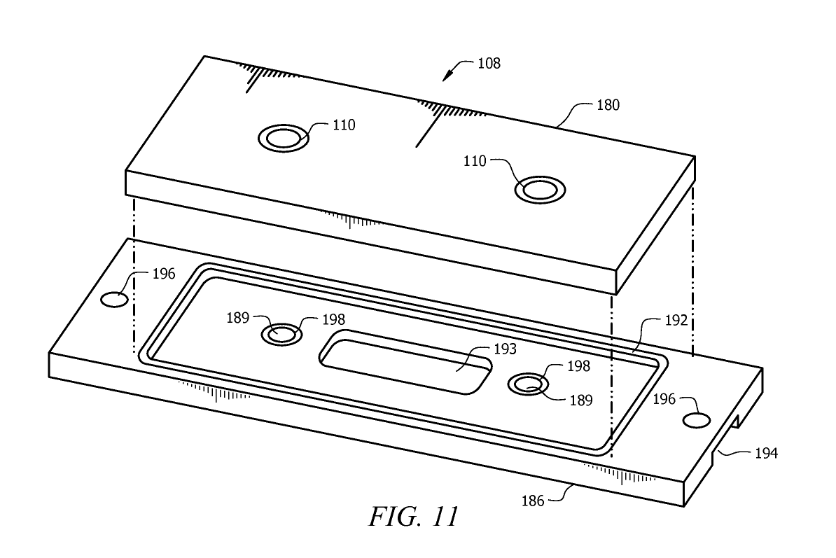

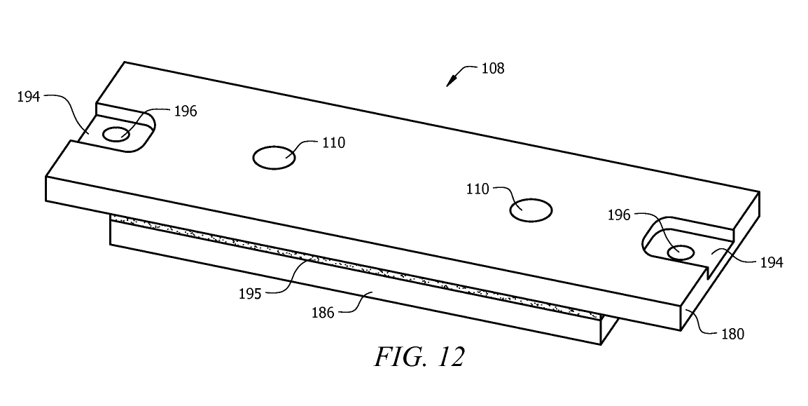

With reference to FIGS. 10, 11 and 12, an embodiment of an implantable device 108, e.g., an implantable neurostimulation device, is depicted before device assembly and after device assembly. Specifically, FIG. 10 illustrates an embodiment of a pre-assembled implantable neurostimulation device, FIG. 11 illustrates the pre-assembled implantable neurostimulation device from a reverse perspective, and FIG. 12 illustrates the assembled implantable neurostimulation device. A ceramic base 180 may be formed of alumina including metallizations to electrically connect and contact components of the implantable device 108. The ceramic base 180 may electrically connect the internal coils 182, a power management integrated circuit 184, such as an application specific integrated circuit (ASIC), and the power storage capacitor 188. A ceramic cover 186 may be formed of alumina including metallizations to electrically connect the neurostimulation electrodes 110 to contacts 198 on the ceramic cover 186. The ceramic cover 186 may include a component cavity 193 to provide space for the components on the ceramic base 180 when the ceramic cover 186 and the ceramic base 180 are joined.

Multilayer ceramics may be used for implantable medical devices requiring RF energy transmission. A multilayer ceramic is a material constructed by laminating several layers of unfired (or green) ceramic material together or by laminating alternating layers of unfired ceramic material and metal together. The laminated assembly may be fired together (i.e. co-fired) such that the laminated assembly becomes one entity in order to form the final multilayer ceramic material. Multilayer ceramics are substantially nonmetallic, allowing RF signals to pass where there is no metal embedded within the ceramic structure for the purposes of power and/or communication. Multilayer ceramics are biocompatible if they are made from predominantly biocompatible ceramics such as alumina (Al₂O₃) and very little or no non-biocompatible materials are exposed to the body. Multilayer ceramics may have various features cut into each layer in order to form complex geometries, such as the component cavity 193, fill holes 196 and/or connector slots 194 for physically connecting multiple devices.

Embedding metal within the ceramic to create a conduction path between the device circuitry (chip, ASIC, etc.) and the stimulation electrode 110 introduces a potential leak path for fluids from the exterior of the device to that circuitry (i.e. a potential path to lose hermeticity). In traditional packages using fired ceramics or metal enclosures, these leak paths may be sealed using processes such as ceramic-to-metal brazing, metal-to-metal brazing, etc. to fill the interface between the electrode 110 and the device exterior wall with a nonpermeable material such as a metal or metal-ceramic composite. This extra step can be avoided using multilayer ceramic technology. The conduction path between the circuitry and external electrodes 110 in a multilayer ceramic are created by punching holes in the unfired ceramic and filling the holes with a refractory or high temperature melting material such as tungsten, molybdenum, molybdenum-manganese alloy, etc. When the ceramic is fired to harden its structure, the joint between the ceramic and the metal may become hermetic as the ceramic shrinks onto the metal during firing.

The metallizations within the ceramic base 180 and ceramic cover 186 are positioned using a multilayer assembly process before the ceramic is fired. The fired ceramic base 180 and the ceramic cover 186 are prepared to be sealed together. A metallization ring 190, such as gold, is positioned around the circumference of the ceramic base 180 to meet a gold-tin solder ring 192 on the underside of the ceramic cover 186. The gold-tin solder ring 192 may be melted or welded directly onto a metallization ring 190 on the cover 186 prior to aligning the base 180 and cover 186. The assembly of the base 180, cover 186 and solder ring 192 are heated under temperature and pressure to melt the solder, forming a hermetic seal between the base 180 and cover 186, resulting in a hermetic ceramic housing 464 (as shown in FIG. 9 above) for the contained components. Alternatively, the gold-tin solder ring 192 could be shaped as a separate frame and assembled into place onto the base 180 or cover 186 in FIGS. 10, 11, and 12. Alternatively, the gold-tin solder ring 192 may be melted or welded directly onto a metallization ring 190 on the base 180 and meet a metallization ring 190 on the cover 186. Alternatively, the solder could be lead-tin or another alloy with a melting temperature below about 400° C. to ensure that temperature sensitive components, such as the capacitor 188, still function as intended. An 80/20 gold-tin solder is particularly suitable because the high gold content of the solder may provide good biocompatibility properties, and the eutectic nature of the solder allows a low melting temperature (from about 280 to about 320° C.), which may not damage the components within the package, such as the capacitor 188.

Contacts 198 on the base 180 and cover 186 may be electrically joined to connect the electrodes 110 on the cover 186 to the circuitry metallization and components on the base 180. Solder 189 may be welded or melted onto the contacts 198 of the cover 186. The solder may have the same or a lower temperature as the hermetic seal solder ring 192 in order to facilitate assembly of the base 180 and cover 186. If the solder 189 melts at a higher temperature then the solder ring 192, then the solder 189 may cause a gap to form between the hermetic seal solder ring 192 and the metallization ring 190 that it is designed to reflow onto in order to form the hermetic seal. The contacts 198 on the base 180 are aligned to the contacts 198 on the cover 186 simultaneously with the operation of aligning the metallization ring 190 on the base 180 to the solder ring 192. The solder 189 is reflowed onto the mating contact 198 of the base 180 during the reflow of the base 180 and cover 186 with solder ring 192. Alternatively, the solder 189 may be melted or welded directly onto the contact 198 on the base 180 and may meet the contact 198 on the cover 186. Alternatively, a solder preform may be inserted in between the contacts 198 of the base 180 and cover 186. The solder 189 may be gold-tin, lead-tin, or another solder whose melting temperature does not damage the components contained within the housing. The solder 189 may be circular, rectangular, polygon, or ring shaped. The solder 189 may be the same thickness as the solder ring 192. Alternatively, the solder 189 may be thicker than the solder ring 192 if the solder 189 melting temperature is at least about 10° C. lower than then melting temperature of the solder ring 192 such that the solder 189 melts prior to the solder ring 192 melting during the solder ring 192 reflow operation.

A biocompatible covering (or seal) 195 may be set over the seal joining the ceramic base 180 and ceramic cover 186, to increase biocompatibility of the implantable device 108. The actual size of the biocompatible covering 195 has been exaggerated in the figures to make it visible but would typically be of a size appropriate to cover the ceramic joint. The biocompatible covering 195 may be platinum, titanium nitride, iridium oxide, titanium oxide, gold, MP35N alloy as defined by American Society for Testing and Materials (ASTM) Standard F562, 316L stainless steel, etc. The thickness of the coating is typically from about two microns to about 50 microns, depending on the durability of the coating desired.

The hermetic ceramic housing 464 (in FIG. 9) may be assembled using two pieces of alumina ceramic using the multilayer ceramic process. In accordance with an embodiment, the hermetic ceramic housing 464 may contain the base 180 and the cover 186 depicted in FIGS. 10, 11, and 12. Stacking the base 180 and cover 186 may form a smooth exterior surface and an interior volume. The base 180 and cover 186 may be solid and substantially rectangular.

The base 180 may be a flat piece of multilayer ceramic constructed of several alternating layers of unfired alumina and refractory metals that were assembled and then fired together to form a single internally hermetic firm structure. Each sheet of alumina is solid except where holes are punched so that vias may electrically connect metallization patterns between separate layers. The bottom surface of the base 180 contains metallization patterns representative of the electrodes 110 desired on that surface. The metallization is typically a stack of refractory metals (tungsten, molybdenum, molybdenum-manganese, etc.), nickel, and gold. Vias filled with refractory metals connect the metallization patterns on the bottom of the base to an upper layer surface of the base where a separate metallization pattern may form the circuitry for the device. Vias filled with refractory metals may connect the circuit metallization pattern to a further upper layer surface of the base where another metallization pattern may form lands or pads for component attachment. In this manner, the various traces of the circuit pattern are covered by a layer of alumina, eliminating the risk of unintentional shorting between components. The metal to which components attach is typically a stack of refractory metal, nickel, and gold. The upper most surface of the base 180 may contain a seal ring metallization pattern or metallization ring 190 typically made of a stack of refractory metal, nickel, and gold.

The ceramic cover 186 may be a flat piece of multilayer ceramic constructed of several alternating layers of unfired alumina and refractory metals that were assembled and then fired together to form a single internally hermetic firm structure. The top surface of the cover 186 may contain metallization patterns representative of the electrodes 110 desired on that surface. The metallization is typically a stack of refractory metals, nickel, and gold. Vias filled with refractory metals connect the metallization patterns on the top of the cover to a lower layer surface of the cover where a separate metallization pattern may form the circuitry for the device 108. In this case, the circuitry routes the electrical signals to the contacts 198 that will be aligned to similar contacts 198 on the base 180 when the two halves of the housing 464 are assembled. Vias filled with refractory metals may connect the circuit metallization pattern to a further lower layer surface of the cover 186 where another metallization pattern continues this purpose. The lower most layer of the cover 186 will contain the actual lands or pads that form the contacts 198. The contacts 198 are typically a stack of refractory metal, nickel, and gold.

One or more multiple layers of the unfired alumina below the layer to which the cover electrodes 110 are attached may have voids punched within in it to form the component cavity 193 into which the electrical components may fit. On the base 180 or the cover 186, the layer of the unfired alumina containing the electrode metallization may have voids cut within it in order to form an exterior depression in cover 186 or base 180 into which polymer may flow to aid in attaching multiple devices into the array 466 (as shown in FIG. 9 above). FIG. 10 depicts this depression as the connector slot 194 in the cover 186. This depression may extend into other unfired layers below the layer containing the electrode metallization. All layers of alumina may have a hole or void punched through an exterior layer or all layers at the longitudinal ends of the device in order to form the fill hole 196 into which polymer may flow to aid in attaching multiple implantable devices 108 into the array 466 (in FIG. 9). In FIGS. 10, 11, and 12, the lower-most surface of the cover 186 may contain a seal ring metallization pattern or metallization ring 190 typically made of a stack of refractory metal, nickel, and gold.

Components are assembled to the base circuit layer using traditional processes (solder, epoxy, conductive adhesive, etc.) Gold-tin or another solder melting under about 400° C. may be deposited on the cover 186 to form the seal metallization ring 190 and the contact metallization pattern or contacts 198 for connecting the cover electrodes 110 to the base 180 circuitry. Deposition methods include electroplating, molten deposition, and vapor deposition. A solder preform or gold-tin solder ring 192 may be positioned atop the seal metallization ring 190 on the base 180 or cover 186. A solder preform or solder 189 may be positioned atop the contacts 198. The seal metallization ring 190 is typically in the shape of a picture frame. The electrode connection preform is typically in the shape of a disk or rectangle. The base 180 and cover 186 are positioned such that the solder ring 192 and the exposed mating metallization ring 190 align. The two halves are heated to such a temperature to cause the solder to melt and form a hermetic seal. Any exposed metal where more biocompatible properties are desired may be plated, sputtered, or otherwise coated with a substantially biocompatible metal such as platinum or titanium nitride to form the biocompatible covering 195.

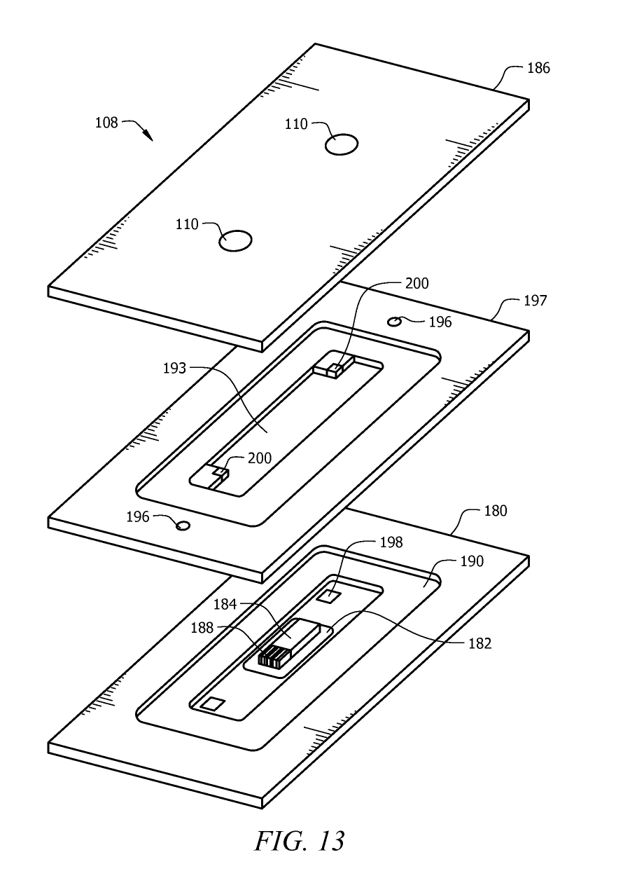

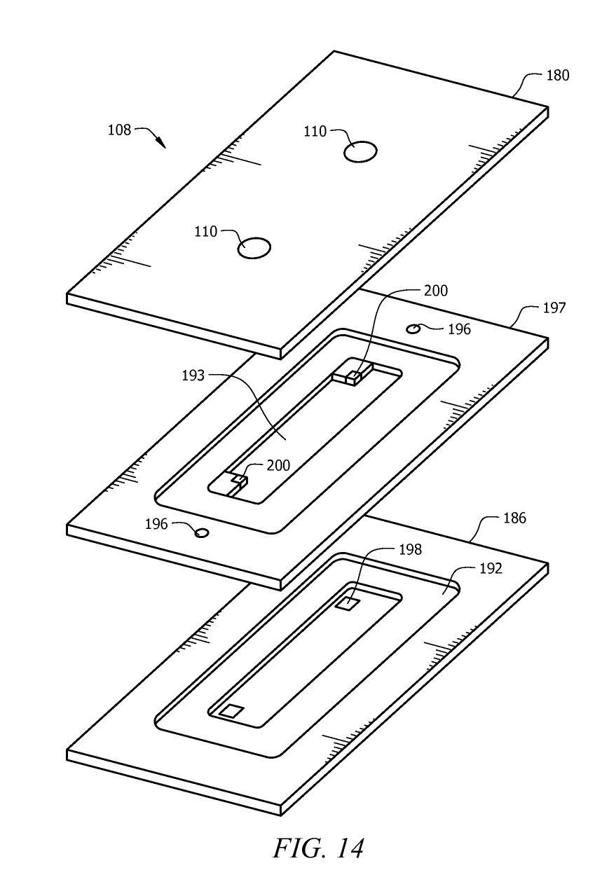

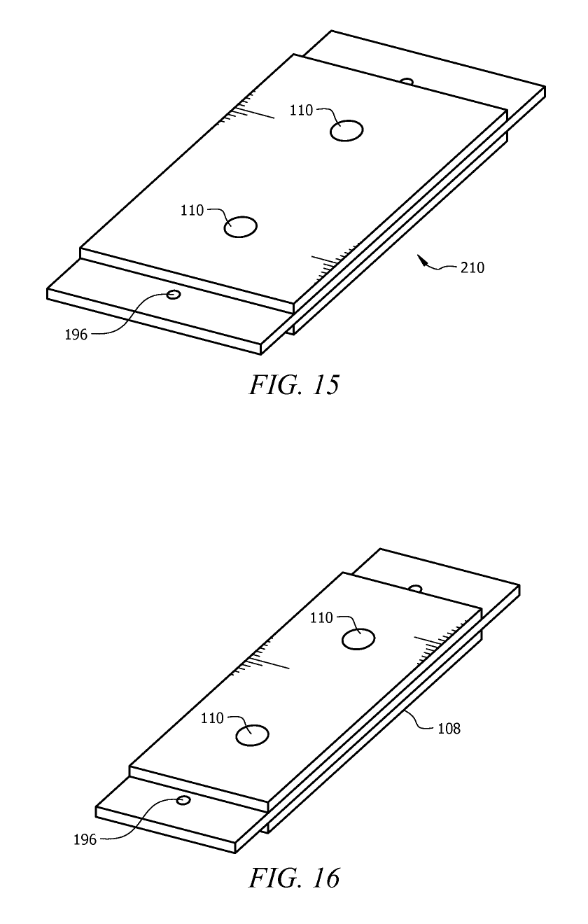

With reference to FIGS. 13, 14, 15, and 16, an embodiment of an implantable device 108, e.g., an implantable neurostimulation device, is depicted before assembly and after assembly. Specifically, FIG. 13 illustrates an embodiment of a pre-assembled implantable neurostimulation device, and FIG. 14 illustrates the pre-assembled implantable neurostimulation device from a reverse perspective. FIG. 15 illustrates the pre-cut assembled implantable neurostimulation device and FIG. 16 illustrates the assembled and cut implantable neurostimulation device. Fired ceramic plates, such as a ceramic base 180, a ceramic intermediate 197, and a ceramic cover 186, may be cut and machined to accommodate the internal components. Metallizations may be set on the surface of the ceramic plates and through the plates to connect the internal components and electrodes 110. A plurality of caps 200 connect the internal components through a filled via to the electrodes 110 on the ceramic cover 186 and the ceramic base 180. In accordance with an embodiment, the ceramic base 180 and ceramic cover 186 are cut shorter than the ceramic intermediate 197 and assembled to form a pre-cut assembly 210. The pre-cut assembly 210 may be cut again to form the housing of the implantable device 108

The base 180 may be a flat piece of ceramic. The ceramic base 180 may be metalized on both sides. One side may contain the circuit layer and a solder ring 192. The other side may contain the metallization for the electrodes 110. Vias through the ceramic base 180 may connect the electrodes 110 to the circuit layer. The vias may be hermetically filled with a metal or metal-ceramic paste in order to eliminate any potential fluid leak paths through the ceramic base 180.

The ceramic intermediate 197 may be an annular rectangle shaped piece of ceramic. Both sides of the ceramic intermediate 197 may be metalized with the solder ring 192. Two vias may be placed interior to the solder ring 192 capped on both sides by metalized lands. The vias are hermetically filled with a metal or metal-ceramic paste in order to eliminate any potential fluid leak paths through the ceramic.

The cover 186 may be a flat piece of ceramic. The ceramic cover 186 is metalized on both sides. One side may contain the circuit layer and the solder ring 192, and the other side may contain the metallization for the stimulating electrodes 110. Vias through the cover piece 186 connect the electrodes 110 to the circuit layer. The vias are hermetically filled with a metal or metal-ceramic paste in order to eliminate any potential fluid leak paths through the ceramic.

The solder ring 192 of the base 180 and intermediate 197 and/or the intermediate 197 and cover 186 may be aligned. Through this process, the lands of the ceramic intermediate 197 may be simultaneously aligned to the appropriate lands of the cover circuit layer or base circuit layer. Solder may be deposited in between the two mating surfaces. This solder deposition may occur before assembly. If solder is used, gold-tin or another solder melting under about 400° C. may be deposited onto either the base 180 or the cover's solder ring 192 and a metallization ring 190 for connecting the cover electrodes 110 to the base circuitry. Deposition methods include electroplating, molten deposition, and vapor deposition. Alternatively, a solder perform or metallization ring 190 on the base 180 or cover 186 atop the solder ring 192 and the metallization ring 190 may be used for connecting the cover electrodes 110 to the base 108 circuitry. The solder ring 192 is typically in the shape of a picture frame. The electrode connection preform is typically in the shape of a disk or rectangle. The assembly may be fired to mechanically and hermetically join the two pieces. Components are assembled to the base circuit layer using traditional processes (solder, epoxy, etc.)

The ceramic base 180 and ceramic cover 186 are aligned such that their seal patterns align. Through this process, the non-mated lands of the ceramic intermediate 197 may be simultaneously aligned to the appropriate non-mated lands of the cover circuit layer or base circuit layer. Solder is deposited in between the two mating surfaces. This solder application may occur before assembly. The assembly is fired to mechanically and hermetically join the two pieces, forming a hermetically sealed vessel.

In accordance with an embodiment, the implantable device 108 functions as an electric neurostimulator and includes electrodes 110 on the ceramic base 180 and the ceramic cover 186. At least two electrodes 110 are typically required to provide a voltage difference between them, thereby injecting currents into the adjacent tissue. An electrode 110 may be provided on a ceramic cover 186. A second electrode 110 may be provided on the ceramic base 180. In accordance with an embodiment, two electrodes 110 may be provided on the ceramic cover 186. Two more electrodes 110 may be provided on the ceramic base 180. Current steering may be accomplished by selecting one of the four electrodes 110 as positive and a second of the four electrodes 110 as negative.

In accordance with an embodiment, a neurostimulator may have two electrodes 110 on the ceramic cover 186 and two electrodes 110 on the ceramic base 180. All four electrodes 110 may be tri-state (positive, negative, off), and each electrode 110 may be polarized individually. By selecting the polarity of the electrodes 110, the resulting currents can be steered, for stimulation that is more effective.

In accordance with an embodiment, an implantable device 108 may have four electrodes 110 positioned on two sides of the implantable device 108. The arrangement of electrodes 110 on the sides of the implantable device 108 can be extended to use any number of electrodes 110 on any number of sides of the implantable device 108.

The shape of the implantable device 108, for example, may be a rectangular solid, with two faces significantly larger than the other faces. By placing two electrodes 110 on each of the large faces, the current fields can be concentrated near either large face or around the edges of the faces. Because the proximity of the current fields to the nerve tissue changes the probability of nerve activation, it is possible to direct the current fields to a specific space and enhance the ability to recruit nerve fibers.

The ceramic layers of the implantable device 108 may be hermetically sealed and made biocompatible. In accordance with an embodiment, the ceramic base 180, ceramic intermediate 197 and ceramic cover 186 may be hermetically sealed using a gold-tin solder ring 192. A platinum seam or biocompatible covering 195 may cover over the ceramic joint where the gold-tin solder is exposed to make the device more biocompatible.

In accordance with an embodiment, the implantable device 108 may include electrodes 110 in the form of exposed metallic surfaces. The electrodes 110 may be positioned on the top surface of a device housing 464. Additionally, the electrodes 110 may be positioned on the bottom surface of the device housing 464. In accordance with an embodiment, two electrodes 110 are positioned on the top surface of the device housing 464 and two electrodes 110 are positioned on the bottom surface of the device housing 464.

The ceramic base 180, ceramic intermediate 197, and/or ceramic cover 186 may be machined, cut, or otherwise processed as required to engage each other, to enclose the internal components with sufficient tolerances and as otherwise appropriate. A connector slot 194 and a fill hole 196 may be included in the ceramic cover 186, ceramic base 180, and/or ceramic intermediate 197 to enhance connection to the polymer connectors discussed below.

The electrodes 110 may be connected to charge-balancing circuits. The charge balancing circuits may be passive, as in a resistance connected between the electrodes 110. The charge balancing circuits may be active, as in a switching element connected between the electrodes 110. The electrodes 110 may be circular in shape, with a diameter of from about 0.25 to about 3 millimeters. The electrodes 110 may be spaced from about 2.5 to about 6 millimeters apart. In accordance with an embodiment, the electrodes 110 may have a diameter of about one millimeter and be spaced about four millimeters apart.

In accordance with an embodiment, polymer is used to cap the end of the implantable device housing 464. Suture holes or other anchoring devices may be positioned in the polymer. The polymer may be used as a lead to connect the implantable device housings 464 into a chain or the array 468. Polymer may be used to set the implantable devices 108 a determined distance apart. Polymer may also be used to encapsulate the entire implantable device 108, with the exception of the electrodes 110.

In accordance with an embodiment, the ceramic base 180 and the ceramic cover 186 are metalized and sealed with solder to form a hermetic seal. Alternatively, the ceramic base 180 and the ceramic cover 186 are metalized and sealed through a brazing process to from a hermetic seal. Alternatively, the ceramic base 180 and the ceramic cover 186 are metalized and brazed to metal lead frames which are in turn laser welded, brazed, or resistance welded to form a hermetic seal. Alternatively, the ceramic base 180 and the ceramic cover 186 are metalized and may be ultrasonically vibrated together to form a hermetic seal.

In an embodiment, a biocompatible ceramic base substrate or base 180 may be metalized using thick film, thin film, or physical vapor deposition processes with the circuit traces required by the device. The substrate may consist of alumina from about 90 to about 99.9 weight percent (%) purity or other biocompatible ceramics. The substrate may have a thickness from about 0.005 inch to about 0.5 inch.

The circuit metallization may be capped with one or more solderable/brazeable materials appropriate to the method of component attachment. Examples of such metals include silver, nickel, palladium, gold, gold alloys, silver alloys, palladium alloys, etc. The thickness of this metallization layer may be from about 10 micrometers or microns (μm) to about 500 μm. A barrier metal of nickel, palladium, etc. may be under the solderable metal to prevent the solderable metal from dissolving into the solder during the soldering or brazing process. The thickness of this metallization layer may be from about 10 μm to about and 500 μm. Where appropriate, the circuit metallization may be connected to hermetically sealed vias that travel through the substrate to circuit pads on the opposite side of the substrate.

Circumscribing the circuit metallization may be an unbroken metallization ring 190 that is attached to the substrate. The width of this metallization may vary from about 0.005 inch to about 0.250 inch. This perimeter metallization may be capped with an appropriate metal such as gold from about 10 μm to about 500 m in order to form a solder wettable surface. Atop this wettable surface may be deposited a layer of solder ring 192 such as 80% gold and 20% tin in a thickness of from about 0.001 inch to about 0.050 inch. The solder width is typically from about 10 to about 50% narrower than the width of the metallization ring 190 in order to accommodate proper solder flow during the solder reflow process. The various components may be placed on top of the circuit metallization at the appropriate circuit pad locations using standard processes such as solder, conductive epoxy, solder balls, ribbon bonding, etc., to construct the working circuit that constitutes the electronics of the implantable device 108.

Separately, a biocompatible ceramic with a similar coefficient of thermal expansion to the before-mentioned ceramic base substrate or base 180 may be constructed to form the ceramic cover 186 of the implantable device 108. The ceramic cover 186 may have a cavity in one surface to accommodate the electronics protruding from the ceramic base 186. Around the perimeter of this ceramic cover 186 may be a pattern of solder wettable metallization ring 190 that mimics the shape of the perimeter metallization ring 190 on the base 180. The cover's metallization ring 190 may be wider or narrower than the base metallization ring 190 in order to compensate for assembly tolerances and proper solder flow during the solder reflow process. Atop the cover metallization ring 190 may be a solder ring similar to as described for the base 180.

The ceramic cover 186 may be placed atop the base 180 such that the electronics are within a component cavity 193 on the cover 186 and the perimeter metallization ring 190 and the solder ring 192 align. The ceramic cover 186 and ceramic base 180 may be heated under pressure to a temperature from about 100° C. to about 400° C. to allow perimeter solder to reflow, hermetically sealing the two substrates together. All potential non-biocompatible metal surfaces such as the exterior of the perimeter solder, perimeter solder, solder wettable metallization, and/or exterior of the vias may be electrolessly or electrically electroplated with platinum or gold to assure biocompatibilty of the implantable device 108.

In accordance with an embodiment, the electrodes 110 may be made of platinum or a platinum-iridium alloy. The electrodes 110 may be attached to the metallizations on the exterior of the implantable device housing 464 using a variety of known methods, including sputtering the electrode 110 onto the housing 464, or attaching an electrode 110 using ultrasonic welding, or by using a conductive paste or epoxy. The electrodes 110 may be attached by electroplating platinum or platinum/iridium onto a preexisting metallization pattern on the base 180 and cover 186 in the shape of the electrode 110 to be created. The electrodes 110 may be attached by printing a substantially platinum or platinum/iridium paste onto the fired ceramic surface where desired. The printing may completely cover the electrode metallization pattern created on the surface of the base 180 and cover 186. The metallization pattern on the cover 186 or base 180 may be smaller than the final desired electrode shape and may be limited to the size of the filled via connecting the electrode 110 to the circuit layers of the substrates. The electrodes 110 may be manufactured by gold brazing a platinum or platinum/iridium machined electrode 110 onto the electrode metallization pattern of the base 180 or cover 186.

The electrodes themselves may cover the exposed metallization and vias on the base 180 or cover 186 which themselves may not be biocompatible. Thus, the biocompatible electrodes improve the biocompatibility of the device 108 by covering these potentially non-biocompatible features. The electrodes 110 may be manufactured by placing a sheet of the desired electrode metal (e.g. platinum, gold, MP35N, etc.) that may be between 0.001 inch and 0.25 inch thick on top of the metallization pattern for the electrodes on the base 180 or cover 186. The electrode material may be biocompatible and have the electrical characteristics desired. The sheet of electrode metal is ultrasonically welded to the via. Because ultrasonic welding is a metallurgical process between the via metal and the electrode metal, many different types of metal may be combined without significant heat build up, intermetallic formation, or adverse affect on the surrounding ceramic. An ultra violet (UV) laser, carbon dioxide (CO₂) laser, water jet, or other means may be used to cut around the periphery of the desired electrode shape. The excess electrode material may be removed, leaving the electrode 110 exposed and electrically and mechanically connected to the via.

As an alternative embodiment, the ultrasonic welding area may be significantly larger than the via such that the welding zone covers multiple vias and simultaneously welds multiple vias at once.

In another embodiment, the fill metal of the via may be covered by a thin layer (from about 1 μm to about 30 μm) of an intermediary metal such as nickel, gold, etc. to aid in forming the ultrasonic bond between the biocompatible sheet and the via. The electrode 110 and intermediary metal may be plated with a biocompatible material if one was not used as the intermediary metal.

The electrodes 110 may be covered by the same biocompatible material used to cover the solder ring 192. The electrode coating may occur simultaneously with the coating of the solder ring 192 during manufacturing. The biocompatible material may be a non-conductive coating that covers the exposed surface of the metallic electrodes 110. Any non-biocompatible materials exposed during the electrode attachment process may be covered by a biocompatible barrier such as platinum, titanium nitride, iridium oxide, etc.

The electrodes 110 may be completely covered by a suitable biocompatible stimulation electrode material such as iridium oxide or titanium nitride. These materials may imbue desirable electrical properties to the electrode such as high capacitance, reduced impedance, and lower polarization effects in-vivo. This same coating material may be used to cover the solder ring 192 in lieu of or in addition to the biocompatible covering 195.

The vias may be filled with a metal such as tungsten, molybdenum, molybdenum-manganese, gold, stainless steel, MP35N, etc. in a hermetic fashion. The via diameter may be from about 25 microns (0.002 inch) to about 635 microns (0.025 inch).

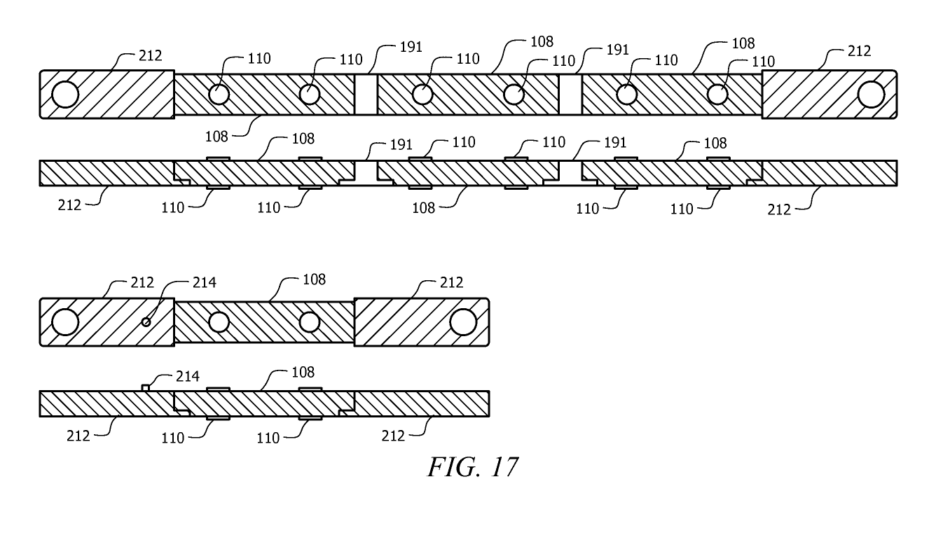

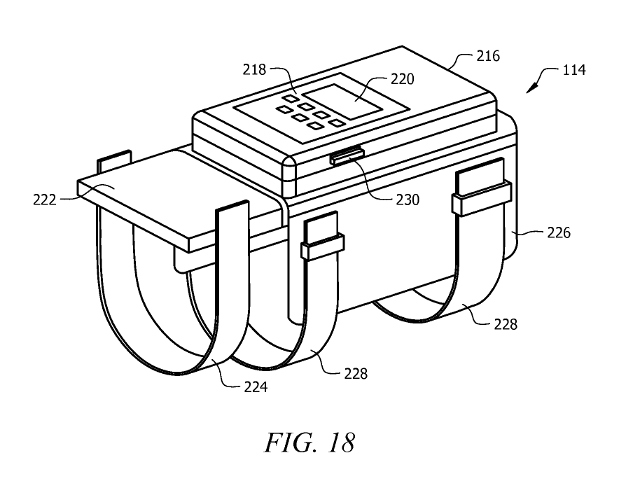

With reference to FIG. 17, a plurality of assembled neurostimulation arrays of implantable devices 108 are depicted. The implantable devices 108 are connected using polymer connectors 191 to form an array. End pieces 212 may be formed of polymer or metal and may provide suture hole connectors or ends. These end pieces 212 may be connected to an implantable device 108 or an array of implantable devices 108. A suture may be passed through the end pieces 212 to anchor the implantable device 108 to appropriate tissue. To identify a particular end or side of the array, an alternate polymer suture hole may identify an end, while a visual orientation indicator 214 may identify a side. A polymer connector 191 may connect two or more implantable devices 108 to form a chain. The distance between the implantable devices 108 may be determined by the length of the polymer connectors 191.

To form a neurostimulator array 468 (see FIG. 9), two individual neurostimulation units or implantable devices 108 may be placed into a mold a predetermined distance apart. The distance may be from about 0.25 mm to about 25 mm apart. Liquid polymer may be injected under heat and pressure into the space between the adjacent units and cured. This process may be a liquid injection molding process. Any number of individual implantable devices 108 may be chained into an array 468. Each array 468 may be capped with suture ends 212. The proximal or distal individual neurostimulation unit or implantable device 108 in the array 468 may be placed into a mold a predetermined distance apart from a pre-manufactured suture end 212 that is also placed in the mold. The distance between the two parts may be from about 0.25 mm to about 25 mm apart. Liquid polymer may be injected under heat and pressure into the space between the adjacent implantable devices 108 and cured. This process may be a liquid injection molding (LIM) process. This process may be repeated for the opposite end of the array 468. Alternatively, an implantable plastic such as polyphynylsulfone (e.g., RADEL), polyether ether ketone (PEEK), or polysulfone may be injected directly onto the outside proximal or distal most surface of the array 468 to form the suture end 212. The polymer material may cure and harden in a shape dictated by the mold into the before mentioned shape.

The suture end 212 may provide a means by which the array 468 may be fixated to adjacent or underlying fascia. The suture end 212 is roughly rectangular or semicircular in shape with a hole through its thickness to allow suture needle passage. The suture end 212 may be metal such as implant grade stainless steel, titanium, MP35N, platinum, etc. in order to aid with fluoroscopic and ultrasound visibility. The suture end 212 may be machined, molded, or cast. One face of one of the two suture ends 212 may be coated with anondization, gold plating, titanium-nickel, aluminum-titanium-nickel, aluminum-titanium-nickel-chromium-nickel, chromium-nickel/chromium-carbon, etc. to give a contrasting color to that surface compared to the bare metal of the uncoated surfaces. Alternatively, one face of one of the suture ends may be laser etched to give it a contrasting appearance as compared to the bare metal of the uncoated surface. The uncoated surfaces may be polished to enhance this contrast. This contrasting face may serve as the visual orientation indicator 214 and the means by which a physician can differentiate the ends of the device. This contrasting face allows the physician to know the orientation of the device. If one surface of a single suture end 212 is colored, then each electrode 110 can be assigned a particular reference designation from that surface. Alternatively, two different colors could be used for a single face of the two suture ends 212.

The suture end 212 may be made of a polymer such as RADEL, PEEK, polysulfone, etc. The suture end 212 may be molded or machined. Polymer suture ends 212 may allow RF and x-ray energy to pass through the part in greater quantities than metal suture ends 212.

The suture end 212 may be made of two differently colored materials so that one face of the suture end 212 is substantially different in color to other surfaces of the suture end 212. The two materials may be joined into a single part by molding half of the part in one material and then molding the other half of the part in a different material. Alternatively, the color contrast could be added by machining the two parts out of cured polymer material that has different colors contained within the material but are separated by a defined plane.

With reference to FIG. 18, an embodiment of an external controller 114 is depicted. The external controller 114 may include an external controller housing 216 that houses the batteries and processing circuits of the external controller 114. The external controller housing 216 may have interface buttons 218, a LCD screen 220, and a USB port or interface 230. The external controller housing 216 may be electrically connected to a power coil assembly 222. An external controller sleeve 226 may physically hold the parts of the external controller 114 in place, typically on the limbs of a patient. A power coil strap 224 and sleeve straps 228 may be used to affix the external controller comfortably in place.

The USB interface 230 may be used to communicate with the clinical programming system or device 116 (see FIG. 1). The external controller 114 may be designed to run patient selected pre-programmed treatment protocol, for example, once a day. The USB interface 230 may be used by the clinician to program the protocols and download operation logs. The USB interface 230 may also recharge the batteries.

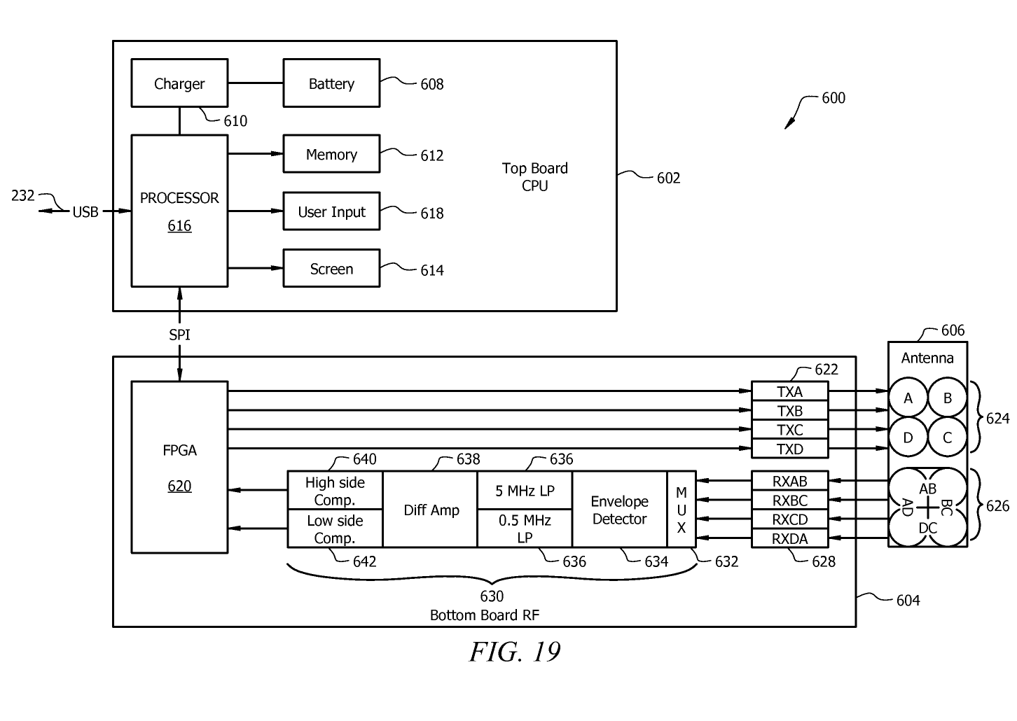

With reference to FIG. 19, a block diagram depicts an embodiment of an external controller circuitry 600. The external controller circuitry 600 may run off a battery 608, for example a lithium-ion battery. A battery charger circuit 610 may charge the battery 608. The battery 608 may be charged whenever the external controller 114 is connected through the USB interface 232 to a personal computer (PC) or to a wall mounted power supply 612 that may include a non-volatile read-only memory (NVRAM), with power being supplied via an identifier (ID) pin on the USB interface 230. For USB powered charging, the rate may be about 100 milliamps and the wall power supply rate may be about 500 milliamps.

A processor 616, e.g., a central processing unit (CPU) may control operation of the external controller 114. Memory 612, such as NVRAM, may provide non-volatile storage for the processor 616. The processor 616 may provide display output using a display screen 614, for example a 128×64-pixel LCD display. The processor may receive input using a user input device 618, such as a keypad, mouse, touch screen, or other data entry device. The processor 616 may be connected to a field programmable gate array (FPGA) 620. Low power board 602 provides a platform for the processor 616.

The FPGA 620 may generate pulse trains to communicate with the implantable device 108, control timing for the receiver circuit 630, and interface with the processor 616. The FPGA 620 may use an external about 81.36 megahertz (MHz) oscillator to generate an about 40.68 MHz signal. The FPGA 620 may be coupled to the processor 616 via a serial peripheral interface (SPI). The FPGA 620 may be part of an RF board 604 that is coupled to an antenna 606.

In accordance with an embodiment, the antenna 606 may be a four layer flex circuit designed to generate a required B-field at the implant depth and at the same time provide detection of field variations caused by the implants shorting their internal receiver's antenna. The antenna 606 may include a single transmission coil 624 connected to the FPGA 620 by a transmission amplifier 622 on the RF board 604. Changes in the load on the antenna 606 may be detected by data detection circuit 630. The antenna 606 may include four transmission coils 624, connected to the FPGA 620 by transmission amplifiers 622 on the RF board 604. The antenna 606 may also include four data detection coils 626, connected to data detection circuits 630 by receiver buffers 628 in the RF board 604. The transmission amplifiers 622 may be quad push-pull Class E amplifiers. The FPGA 620 drives bipolar transistor gates in the correct sequence to generate the required current. The transmitter may send a pulse by reverse phasing the drive signals, which forces the oscillation amplitude to momentarily decrease to zero.

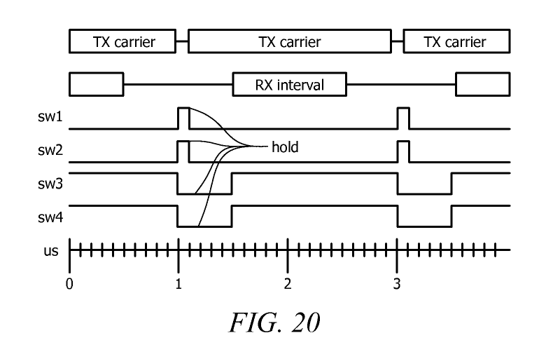

A plurality of detection coils 626 in the antenna 606 are connected to the receiver buffers 628. The timing of the associated signals is illustrated in FIG. 20. The carrier may be comprised of an about 40.68 MHz signal with about 100 nanosecond (ns) nulls occurring every about 2 μs. The null intervals may be the clock pulses used to synchronize data transfer between the external controller 114 and implantable device(s) 108. The implantable device(s) 108 may only respond during a short interval between the clock pulses. Fundamentally, the circuit may be an envelope detector combined with filters and sample/hold timing controlled by the FPGA 620.

The data detection circuits 630 may include a multiplexer (MUX) 632, an envelope detector 634, one or more low pass (LP) filters 636, a differential amplifier 638, a high side comparator 640, and a low side comparator 642. The output of the envelope detector 634 may be run through two low pass filters 636 with different break points. The signals may be passed through the differential amplifier 638 to enhance the signal. The signal then may be passed through two sets of comparators 640 and 642 with resistor-capacitor (RC) filters to determine when the receive amplitude exceeds the average amplitude plus or minus an offset value. The bit stream generated by the comparators 640 and 642 may be fed into the FPGA 620 which does a majority voting on the stream within the receive interval to determine if the implantable device 108 responded with a one or a zero.

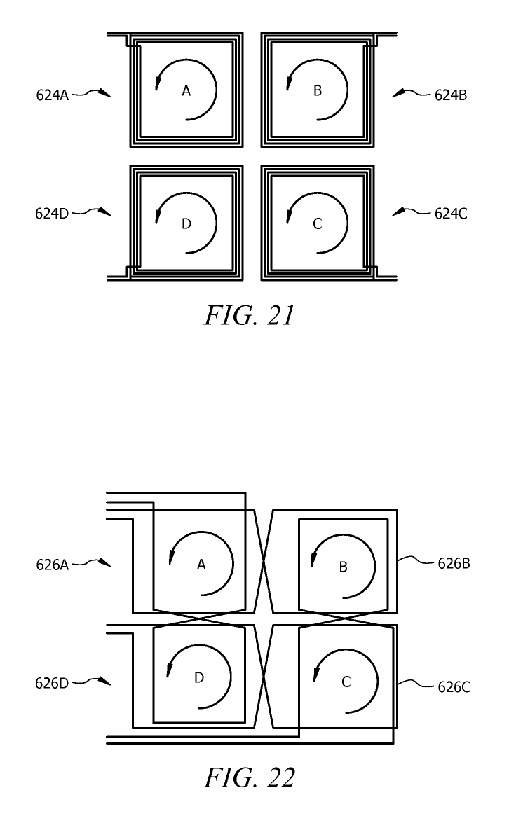

With reference to FIG. 21, four transmission coils 624A, 624B, 624C and 624D (collectively, 624) are depicted in a spiral round configuration and placed in a 2×2 array to cover the treatment area. Each transmission coil 624 may have a dedicated full bridge class E transmission amplifier 622. The coil arrangement and the expected current flow direction within each coil are shown in FIG. 21. This current flow may generate a synchronous B field. Matching current flow direction may be a factor in the receiver operation that is described later.

There are four detection coils 626A, 626B, 626C and 626D (collectively, 626) that may be configured as shown in FIG. 22. Each detection coil 626 may loop around one transmission coil 624 (see FIGS. 19 and 20) and then reverse loops around another transmitter coil 624. If the B fields are equal, then the B field seen by the receiver coil may be zero. The receiver coil may only see the differences between the two B fields.

Returning to FIG. 19, the data detection circuits 630 may include a multiplexer 632. The multiplexer 632 may select which detection coil 626 to listen to for implant responses. Individual implantable devices 108 may be located under two different detection coils 626. The processor 616 can interrogate the implantable device 108 and determine which B-field/transmission coil 624 the implantable device 108 is associated. An envelope detector 634 receives signals from the multiplexer 632 and passes appropriate signals to low pass filters 636. Two low pass filters 636, for example at about five MHz and about 0.5 MHz, may feed into a differential amplifier 638. The high side comparator 640 and the low side comparator 642 may provide output to the FPGA 620.

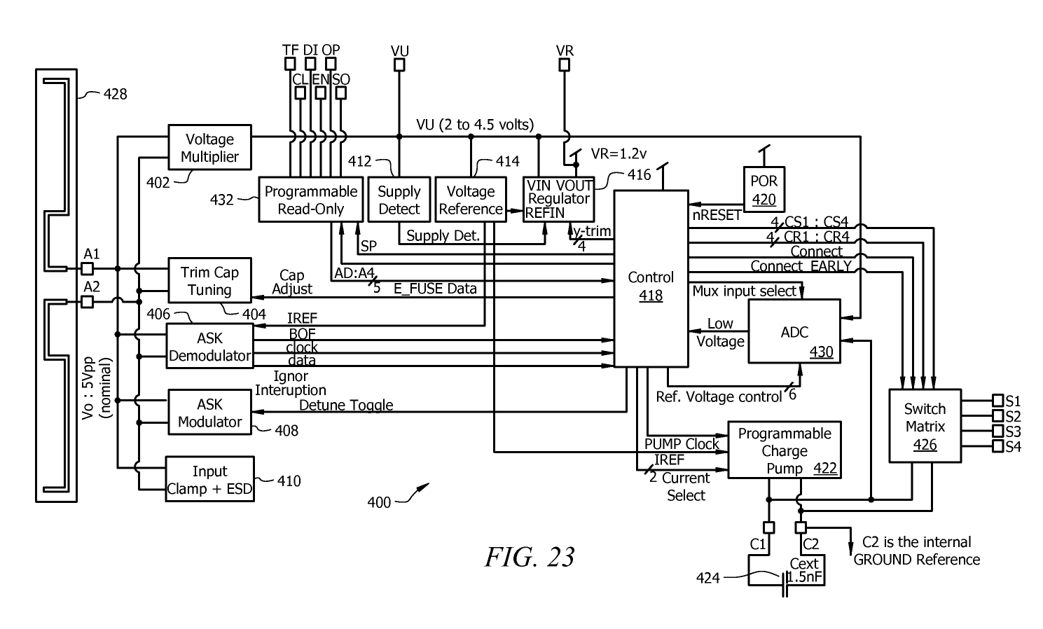

With reference to FIG. 23, a block diagram depicts an embodiment of an integrated circuit 400 that may perform the functional processing of the implantable device 108. The integrated circuit 400 is connected to an internal power and data coil 428. In the integrated circuit 400, a voltage multiplier rectifier circuit 402 may convert an input alternating current, nominally about five volts peak to peak, to unregulated voltage from about 2 volts to about 4.5 volts. A trim capacitor (cap) tuning circuit 404 may be used to fine-tune the device input reactance to match the external antenna for example, at an about 40.68 MHz operating frequency. An amplitude-shift keying (ASK) demodulator 406 may extract clock and data information from modulations on the incoming energy. An ASK modulator 408 may modulate the load of the implantable device 108. In accordance with an embodiment, the ASK modulator 408 may communicate to the ASK demodulator 406 in anticipation of modulation, so that the modulations from the ASK modulator 408 are not received as communications by the ASK demodulator 406.

An input clamp/electrostatic discharge (ESD) unit 410 may limit the received power to prevent overvoltage level on a chip and protect the device from electrostatic discharge events. A supply detection circuit 412 may open when the unregulated voltage level exceeds a preset threshold, for example about 3.1 volts±about 0.0186 volts. The supply detection circuit 412 may include hysteresis control that may not shut down unless the unregulated supply falls below about 1.8 volts±about 0.18 volts. A voltage reference 414 may generate a temperature dependent voltage reference to be used by a voltage regulator 416. The voltage regulator 416 may generate a regulated output voltage for use by internal circuits, for example, a target regulated voltage level might be about 1.2 volts±about 0.12 volts. A control logic circuit 418 may include input control registers, status registers, and operational state machines. A power on reset (POR) circuit 420 may generate a RESET when power levels are unstable, until power is stabilized, for example, an about 5 microsecond (μsec) forced RESET after regulator powers up. A programmable charge pump 422 may charge an external stimulus capacitor 424 to a programmed voltage level. A switch matrix circuit 426 may reverse the polarity of the stimulus output. An analog to digital converter 430 may convert voltage levels to digital signals for processing by the control logic circuit 418.

The operation of the integrated circuit 400 may assume that any incoming RF signal is ramped slowly. The external controller 114 (see FIG. 1) may interrogate the implantable device 108 for supply status. The implantable devices 108 are typically implanted at a consistent depth such that the variance on the incoming power is less than about 20%.

When the integrated circuit 400 initially receives power, a start-up sequence may be started. An external controller 114 (see FIG. 1) may transmit low level RF energy. After about 5 milliseconds, for example, the external controller 114 may interrogate the implantable device 108 for status. The external controller 114 may incrementally increase the RF power until the expected implantable devices 108 respond. When the incoming power reaches a sufficient level, the voltage regulator 416 may be enabled. A low-voltage direct current (DC) may be applied to all internal currents, and the implantable device 108 may respond to the external controller 114 with its status.

The integrated circuit 400 may perform power management functions. A first set of power management functions may be categorized as front-end functions. The front-end functions may include rectification, typically using a rectifier circuit to transform AC into DC voltages. In accordance with an embodiment, a voltage multiplier rectifier circuit 402 rectifies both the positive and negative phases of the incoming AC voltage. This DC level may be the unregulated supply voltage level. The received energy may be of sufficient potential such that the available power can sustain the normal operation of the complete chip. The front-end functions may also include supplied voltage level detection, typically using a supply detection circuit 412 to monitor the unregulated voltage levels from the voltage multiplier rectifying circuit 402 and determine if the voltage is sufficient for normal operations.

When the implantable device 108 is initially powered for operation, the unregulated supply voltage may need to attain a level of about 3.1 volts±about 0.186 volts (DC). Once the supply detect circuit 412 detects a sufficient unregulated supply voltage level, the supply detect circuit 412 may initialize the remainder of the implantable device circuits. When power is initially received, the voltage multiplier rectifying circuit 402, the supply detection system 412, and the voltage reference 414 may be operational.

In accordance with an embodiment, the supply detection circuit 412 may include hysteretic behavior control. Although it may take an unregulated supply level of about 3.1 volts to trigger the assertion of this signal, the unregulated supply level may fall below about 1.8 volts before this control signal is de-asserted. This wide range may allow consistent device operation as the unregulated supply level sags during various operational load conditions.

With the regulator operational, the regulated supply voltage may begin to rise at a rate based on the internal capacitance. The POR circuit 420 may hold the logic in a RESET state for about 5 microseconds after a direct current voltage (VDC) exceeds a potential of about 0.8 volts.

Programmable read-only devices 432, for example a one-time programmable (OTP) memory or fuses, may program a unique 20-bit ID code. Additionally, fuses may be used to hold component trim settings, which may reduce critical component variation.

The external controller 114 may apply a continuous stream of clock and data to all implantable devices 108, such as implanted transponders. During the transponder implant process, a list may correlate each transponder unique identification code with its implanted location.

With the release of the POR signal and completion of electronic fuse (e-fuse) data transfer, the control logic circuit 418 may become active and monitor control input registers. An about 500 kilohertz (KHz) clock, derived from the periodic interruptions of the RF carrier, may be used to clock all internal controller state machines as well as capture the incoming stream of control data from outside the circuit. A shift register may be used to store the incoming bits in the order received. After receiving and shifting in each new bit, the controller may check the input for two things: (1) Validity of the input control word; and (2) The address bit supplied as part of the input control word matches either the unique ID code or the universal code. Specifically, a unique ID code may be used to address each of the implantable devices 108 or transponders. A universal code may be used to address all the implantable devices 108 or transponders. If both checks are TRUE, an address field is used to steer the data to the right location and a data field is loaded to the addressed register. Regardless of the Cyclic Redundancy Check (CRC) result, the implantable device 108 may respond to any transmission that contains a valid unique address.

The front end functions may also include tuning, typically using the trim cap tuning circuit 404 to adjust the resonance of the coils. In accordance with an embodiment, the trim cap tuning circuit 404 employs a set of capacitors that can be added into a receiving circuit, to change the resonance of the receiving circuit. Using feedback, the optimal capacitance can be added to the receiving circuit, to maximize the received power.

In accordance with an embodiment, the analog front end of the integrated circuit 400 can be tuned over a range from about 3.3 picofarads to about 7.175 picofarads. One purpose for this tunability is to provide a means of compensation for the normal manufacturing tolerances of internal and external components. A register may be used to adjust the input capacitance value. The external controller 114 can adjust these capacitor settings to maximize the input voltage level or equalize the unregulated voltage level of multiple implantable devices 108.

The front-end functions may also include over-load protection, typically using the input/ESD unit 410, such as an over-load protection circuit, to measure the in-coming power level and disconnect the rest of the circuit from the received power, or shunt the incoming power away from the other circuitry, in the case where the power level is too high for safe operation.

The integrated circuit 400 may monitor the voltages on the unregulated supply node and the stimulus capacitor. These voltage measurements are made through successive comparisons of the selected node voltage and the value programmed into a 6-bit digital-analog converter (DAC) reference.

The integrated circuit 400 may also include communication functions, typically using the ASK modulator 408 to modulate data to an out-going power transfer and the ASK demodulator 406 to demodulate data from in-coming power transfer.

In accordance with an embodiment, a half-duplex data communication protocol may be implemented. Half duplex is a bidirectional capability communication that is sequential in nature. The external controller 114 and the implantable device 108 take turns transmitting data. ASK may be used as modulation for the transmission of control data to the implantable device 108. In accordance with an embodiment, ASK may be used to send status information back to the external controller 114. ASK transmits information by altering the amplitude of a carrier signal to which a receiver is tuned.

In accordance with an embodiment, the transmission of control information to an implantable device 108 may be accomplished by altering the amplitude of a carrier with a pattern of data. Additionally, by altering the period of the carrier amplitude shift, additional information can be sent. A carrier signal may be interrupted periodically. This periodic interruption may serve as an input clock.

A single interruption may indicate the transmission of a data bit with a state of logical zero. Two interruptions separated by carrier may indicate the transmission of a data bit. Data may be transmitted when the external controller 114 is not attempting to load control information into the implantable device 108.

In accordance with an embodiment, transmission of status information from the implantable device 108 may be a modified form of ASK. The implantable device 108 may transmit data to the external controller 114 when explicitly instructed to do so. An instruction may be transmitted to the implantable device 108 and about three data periods of information may be transmitted.

After the external controller 114 transmits the status request instruction to the implantable device 108, the external controller 114 may continue to transmit the interrupted carrier with a data pattern of all zeros. The implantable device 108 may use a technique referred to as coupled load modulation communication to send the status information to the external controller 114.

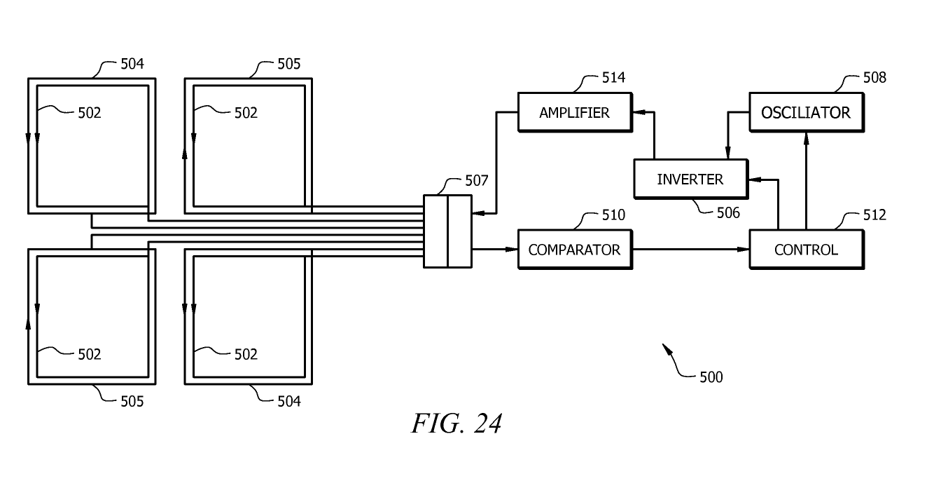

Communication may be accomplished by different methods, including coupled load modulation, backscatter, or generation of harmonic waves. Coupled load modulation may be accomplished by changing the impedance of the implantable device 108 by shunting the antenna terminals for the period of the data bit. In accordance with an embodiment, a low resistance (e.g., about 10 ohms) transistor is closed between the terminals of the antenna to achieve a shunt. This shunt effectively changes the load away from the value that maximized the transmission of power. In accordance with an embodiment, a capacitor can be used as a shunt to change the reactance of the device. The external controller 114 can detect this change of impedance on receiver coils 504 and 505 (see FIG. 24) and may interpret the timing of the impedance changes as data bits.

The coupled load modulation transmission may take place approximately at the midpoint between the normal periodic interruptions transmitted by the external controller 114. The status data may occur over successive data periods. Each status bit may be indicated by an interruption of the carrier. It should be noted that only the first transmitted bit is needed for a successful information packet to be sent and may always be present when the transponder, e.g., the implantable device 108, is functional.

In accordance with an embodiment, a group of registers may be implemented to control the implantable device 108. In accordance with an embodiment, about seven registers may be used to control the operations of the integrated circuit. Each control register may include 8 bits of control data. Initially, all control register instructions may be loaded using a 31-bit control transmission sequence, not including the bits of freedom (BOF) or CRC bits. The control word that is transmitted may be comprised of five segments: (1) a BOF symbol may be transmitted; (2) a twenty-bit ID preamble; (3) a three-bit register address; (4) an eight-bit data byte; and (5) a six-bit CRC Error detection code.

Each transponder 108 may be programmed during manufacturing with a unique ID. This ID may be recognized as part of the input control sequence for the implantable device 108 to load the control information into the internal registers.

All implantable devices 108 may be programmed to recognize a universal 20-bit ID code. The universal ID code may be recognized only for updating the control register and may provide for a convenient method to synchronize the discharge of all implanted device 108. A local ID can be programmed into and enabled by the external controller 114. By reducing the length of the ID code, transmission efficiency may be increased. The local ID code may be enabled by setting a bit in the control register. A CRC code, for example, may be used for detection of bit errors in the unique ID, address, and data fields of the control sequence transmission.

The power management functions may include regulation, typically using a regulator circuit to convert the transmitted RF energy to an appropriate DC voltage level to power the integrated circuits. A regulator may regulate the unregulated supply voltage to an about 1.2-volt DC level, which is used to supply operational energy to many of the implantable device circuits.

The power management functions may include voltage level shifting, typically using the charge pump 422 to increase the regulated voltage to levels sufficient to provide electrical neurostimulation. The amount of charge loaded on the power storage or external stimulus capacitor 424 is specified by selecting a desired voltage to which the external stimulus capacitor 424 may be charged. A stimulus potential register may be used to set this target capacitor voltage.

Internal voltage monitoring may be used to detect the condition when the power storage or external stimulus capacitor 424 charge has reached its programmed value. Once the desired potential has been reached, the control block may gate off the clocks used to run the charge pump 422. The charging of the external stimulus capacitor 424 places a burden on the unregulated voltage supply on the implantable device 108. If the stimulus pulse is to be applied at a maximum rate, the full-allocated current may be drawn from the internal unregulated supply to charge the external stimulus capacitor 424. However, if a slower stimulus rate is needed, it is not necessary to draw the same amount of current. Consequently, a register has been provided that allows the user to select a slower charge current for slower stimulus rates. The stimulus control register may provide two control bits to allow the scaling on the current draw during charge pump operation.

The power management functions may also include a power storage typically using the external stimulus capacitor 424 to store received power, particularly in anticipation of using the stored power for electrical neuro stimulation.

The integrated circuit 400 may include delivery control, typically using delivery control circuits such as the switch matrix 426 to control the delivery of energy to the four tri-state electrodes 110.

The switch matrix 426 may be used to connect the external stimulus capacitor 424 to the stimulus port. The switch matrix 426 may be designed to allow any possible configuration of connection between two input capacitor ports and four output stimulus ports based on the settings of a register. An integrated high impedance path may be always connected in shunt with the selected stimulus ports. The capacitor connection to each stimulus port may be a low impedance path. The maximum current surge during this discharge may be determined by the load impedance. The discharge may be an exponential decaying pulse defined by the time constant of the stimulus capacitor and the load impedance.

A switching system may be used to assign the polarity of each of the electrodes 110. The switching system could respond to instructions or use a closed-loop sensing system to determine optimal polarity assignments.

With reference to FIG. 24, an embodiment of a transmission/receiver system 500 of the external controller 114 is shown. An arrangement of transmission coils 502 and receiver coils 504 and 505 are detachably connected to the transmission/receiver system 500 by a connection component 507. The signal path of the coils 502, 504, and 505 are indicated by arrowheads on the coil drawings. In accordance with an embodiment, the coils 502, 504, and 505 are shaped as about one inch squares.

In accordance with an embodiment, the transmission coils 502 may be several feet distant from the circuits of the transmission/receiver system 500. In accordance with an embodiment, the receiver coils 504 and/or 505 may cover the same area as the transmission coils 502. Receiver coils 504 have a signal path in the same direction as the transmission coils 502. Receiver coils 505 have a signal path in the opposite direction as the transmission coils 502. The receiver coils 504 and 505 may be arranged so that adjacent coils alternate signal path direction. The transmission/receiver system 500 may include a control system 512.

An oscillator 508 may provide a determined frequency, set by the control system 512, to an amplifier 514. A phase inverter 506 can be controlled by the control 512 to change the phase of the signal, e.g., for communication purposes. A comparator system 510 may compare the signals received by the receiver coils 504 and 505, e.g., to detect incoming communication. In accordance with an embodiment, receiver coils 504 and 505 may be high impedance inductor-capacitor (LC) circuits with an area less than the transmission coil 502.

Returning to FIG. 9, the implantable device 108, e.g., an implanted electric-neurostimulator, may include a multi-layered flat coil assembly to receive energy, an integrated circuit, and four tri-state platinum electrodes 110 on opposite faces of a housing 464. The implantable device 108 may use near-field power transmission and communication. The implantable device 108 can be individually programmed to provide a stimulation pulse or a stream of stimulation pulses.

In accordance with an embodiment, the implantable device 108 contains no internal power source and has no wired connects to a power or control source. All consumed power is harvested from RF energy transmitted by the external controller 114. As such, the implantable device 108 may shut down immediately when the external RF source is turned off. In accordance with an embodiment, the implantable device 108 receives power from an external controller 114 using near-field RF transmission. A single flat coil may be used to receive near-field power. The number of turns in the coil, the enclosed mean area of the coil, the shape of the coil, and/or the material used to make the coil may affect the amount of power received. Gold, copper, and/or silver may be used to form coils. Multiple coils may be placed on separate planes relative to each other to increase the amount of energy received. The coils may be electrically connected in series to increase the inductance. The coils may be electrically connected in any manner to increase the coil area in the magnetic flux.

The effectiveness of near-field transmission of power depends on the nature of the tissue that must be traversed by the radiated energy. The depth of the implantable device 108 also affects the reception of power. A first coil may transmit RF energy to a second coil at a given frequency in order to power a circuit attached to the second coil and communicate data at the same time. A typical communications scheme between two magnetically coupled coils is to amplitude modulate the signal or ON/OFF key the signal. However, to optimize the power transfer, the coils may be operated at resonance frequency, which in turn causes ringing in the coils after the input signal is changed or turned off. In applications where the RF energy is also powering a circuit, the RF energy cannot be turned off too long or decreased too much, otherwise the circuitry may not be adequately powered.

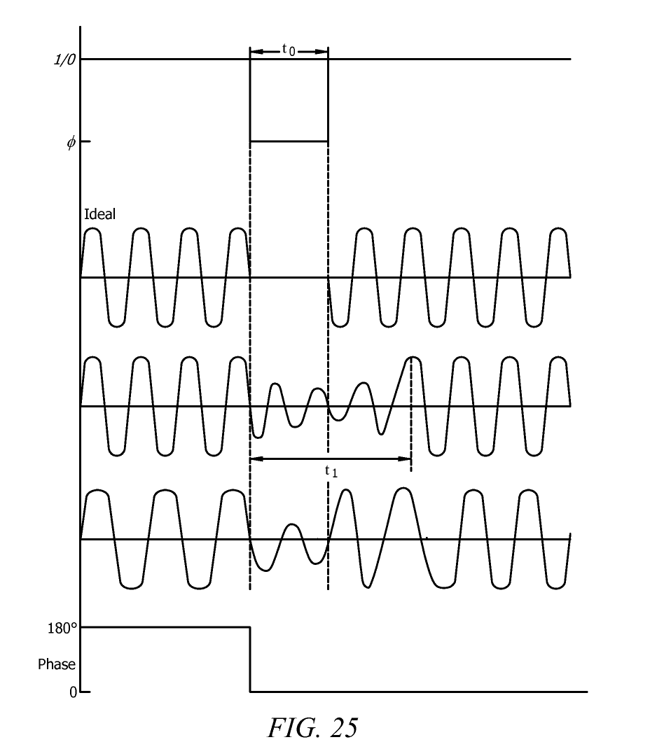

FIG. 25 depicts a series of graphs of communication signals for ringing and phase shift compensation. The coils may cause a ringing in the signal from the step function of the amplitude modulation. Communication may prove difficult due to this ringing. Extending the low voltage period (t₁) during modulation may compensate for the ringing. Extended low voltage periods may cause a loss of power at the load circuit and lower the energy transmission rate. Dampening the ringing allows for higher communication and energy transfer rates. Dampening the ringing may be accomplished by about 180-phase shift of the signal, as shown in the lower part of FIG. 25. This phase shift opposes and dampens the ringing. This dampening may allow for fast recovery time of the signal.

Although phase shifting is another common communication technique, this shift in phase may be what is normally detected, which may require a phase lock loop. This method may use the phase shift to effectively amplitude modulate the signal more quickly than could be done by just changing the amplitude of the signal.

A communication system may provide communication to an implantable device 108 from an external controller 114, using the energy transmitted in near-field power transfer. In normal operation, the external coil may provide power to the internal coil 182. The external coil may transmit a communication signal to the internal coil by reversing the phase of the transmitted power. Using a filter to process the signal received at the internal coil, the implantable device may recognize the presence of a data bit. The phase change may generate a minimal interruption in the transmitted power so that the communication does not disrupt the operation of the implantable device 108. In accordance with an embodiment, a sequence of data bits are transmitted to provide a clock signal to the implantable device 108 and to transmit data such as identification information, parameters, instructions, queries and updates.

The implantable device 108 communicates with the external controller 114 by briefly connecting the leads of the internal coils 182 together. This shorting of the internal coil 182 may form a load modulation that is detected by the external controller 114 using data discrimination coils, e.g., the receiver coils 504, 505 in FIG. 24.

In accordance with an embodiment, two power coils, e.g., transmission coils 502, are driven so that they generate identical B fields. A first data discrimination coil, e.g., the receiver coil 504, is positioned coaxial on a separate parallel plane to the first transmission coil 502. The signal path of the first data discrimination coil 504 is in a first rotational direction, for example clockwise. A second data discrimination coil, e.g., the receiver coil 505, is positioned coaxial on a separate parallel plane to the second transmission coil 502. The signal path of the second data discrimination coil 505 is in a second rotational direction, opposite to the first rotational direction, for example, counter-clockwise. The induced potential in the receiver coil 504 and the data discrimination coils 505 may have equal magnitude and opposite sign if the B field of the transmission coils 502 are substantially identical. A modulated load from an implantable device 108 below the first transmission coil 502 may affect the B field of the first transmission coil 502 but may not affect the B field of the second transmission coil 502. The signals at the receiver coil 504 and the data discrimination coils 505 may not cancel completely and the modulation may be detected.

By including three or more transmission coils 502 and associated the receiver coil 504 and the data discrimination coils 505, the external controller 114 can determine which of the transmission coils 502 has actually detected the coupled load modulation communication. In this manner, coupled load modulation techniques can determine the location of an implantable device 108.

An arrangement of coils 502, 504, 505 may be used to detect load coupling modulated communication from an implantable device 108. This coil design may overcome an obstacle associated with detecting coupled load modulations, which is the fact that a change in impedance from the modulation might go undetected due to the high power required to generate the magnetic field.

The receiver coil 504 and the data discrimination coils 505 may be an even number of coils with the signal direction of half the loops in the opposite direction of the other half of the loops. Any topology consisting of any even number of coils of equal area may be suitable. Another step may be to have an even number of loops with each loop having equal area. Each loop can include multiple turns within each quadrant so that the induced voltage on the loops in multiple quadrants add up to zero when there is no communicated signal. To improve the coupling from the transmitter to the receiver, multiple smaller coils may be used rather than a single large coil. In accordance with an embodiment, four coils may be arranged in a square. These four coils define four quadrants in the overall communication field.

In accordance with an embodiment, the external controller 114 and implantable device 108 communicate using ASK modulation. The data communicated may include timing information and data.

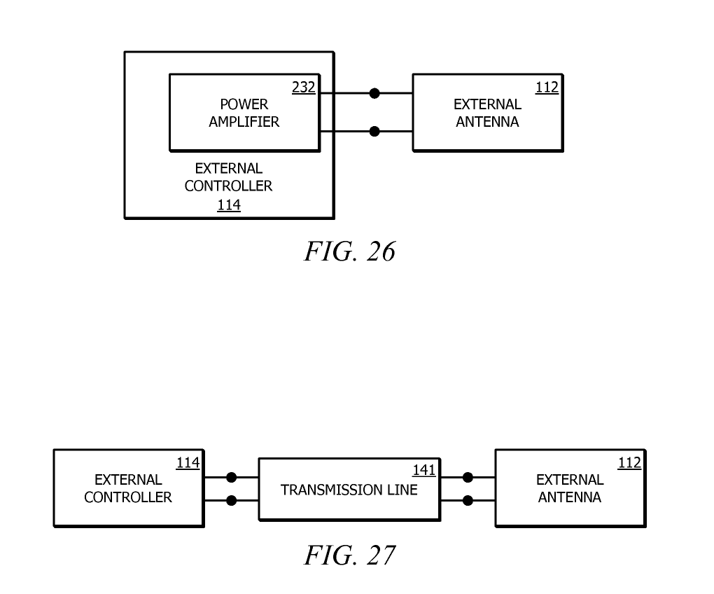

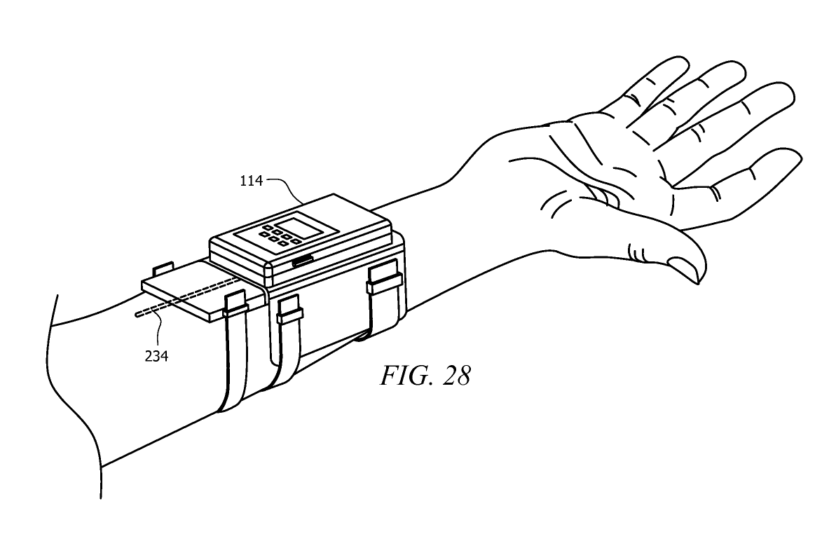

With reference to FIGS. 26 and 27, two embodiments of the external controller 114 are depicted. In FIG. 26, a power amplifier 232 within the external controller 114 is connected directly to the external antenna, so that the external controller 114 and an external antenna, e.g., the external antenna 112, are physically close.

In FIG. 27, the external controller 114 may be connected at some distance to the external antenna 112 by a transmission line 141, for example a coaxial cable. The transmission line 141 may be a coaxial cable, waveguide, wire, optical fiber or any other suitable transmission connection. With a transmission line 141 between the power amplifier 231 (in the external controller 114) and the power coil (on the external antenna 112 side), the external controller 114 may be located away from the body. For example, the external controller 114 may sit upon a tabletop or may be wall mounted. A stand-alone external controller 114 may transmit power including communications using a small antenna and coil located against the skin, for example as a patch, above the implantable device 108.

The inclusion of a transmission cable introduces reactance to the amplifier-coil circuit. As will be recognized by those skilled in the art, the resulting change in the reactance of the circuit can be compensated using other components to resonate at a given frequency. The class-E amplifier typically may include reactive components to provide the tuning of the circuit resonance to the desired frequency. Impedance mismatch may be resolved by incorporating the characteristics of the transmission line into the design of the amplifier.

In accordance with an embodiment, the external controller 114 may be communicably connected to the power coils by a wireless connection. As shown in FIG. 28, the external controller 114 may be strapped on the body at a distance from the power coils. A first antenna, such as a dipole, may transmit power and communications from the external controller 114 to a second antenna connected to the power coils, positioned above an array 234 of implantable device 108. The second antenna receives the power and communications and relays them to the power coils. The power coils transmit power and communications to the implantable device 108. Communications received by the power coils from the implantable device can be relayed to the second antenna and transmitted to the first antenna, for processing by the external controller 114. A programming device, such as a laptop computer, may be connected to the external controller 114 using a USB connection. The programming device 116 may interact with the external controller 114 to set stimulation parameters for the implantable devices 108, such as voltage and frequency.

A variety of software-enabled functions may be processed in the implantable device 108, the external controller 114, and the programming device 116. Identification of an implantable device 108 may be established. Identification of an implantable device 108 can be queried. An identification query may be responded to with identification information. Identification data, such as a local identification code, may be included with communications, instructions, or parameters for a specific implantable device 108. Local identification data may be associated with non-volatile identification data associated with a specific implantable device 108.

Stimulation parameters such as stimulation voltage and stimulation frequency may be established for each implantable device 108 by a clinician using the programmer. The programmer may transfer the stimulation parameters to the external controller 114 where it may be recorded in non-volatile memory. Stimulation parameters are transmitted from the external controller 114 to each implantable device 108 during treatment. The external controller 114 may communicate with each implantable device 108 for each stimulation. The external controller 114 may communicate the frequency and intensity of stimulation to an implantable device 108, which then stimulates at the appropriate frequency so long as the implantable device 108 is powered. The programmer software may respond to communications received from the implantable device 108, in particular to make meaningful changes to the parameters and treatment plan. A wide variety of parameter sets may be available during the testing phase with the doctor and may be selected for transfer to the external controller 114 memory.

The external controller 114 may receive and store parameter settings. The parameter settings may be coordinated for use with therapeutic activities, such as sitting, standing, running, walking, sleeping, eating, writing, or any other suitable activity. Initialization software may be used to record patient data, establish implantable identification data, and set stimulation parameter programs. Operational software may be used to record data during or in relation to therapeutic sessions. Operational software may be used to make changes in the operation of the external controller 114 and/or implantable devices 108. Update software may be used to update the software in the programmer, external controller 114, and implantable devices 108.

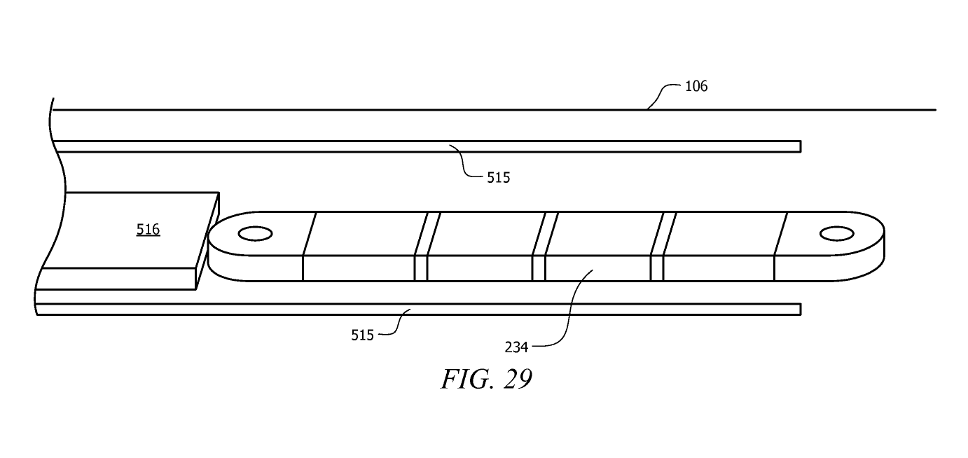

With reference to FIG. 29, a deployed array 234 of implantable devices 108 is depicted. In accordance with an embodiment, the implantation of an array 234 may be performed using a cannula, such as an angiocath. The patient's skin, e.g., the tissue layer 106, is prepared with antiseptic solution and draped to maintain a sterile field. Real time ultrasonography may be used in an array implant procedure to identify and locate nerves and blood vessels. The nerve and blood vessel course may be outlined on the skin with an ink marker. In an embodiment, a 25-gauge 6-inch spinal needle and syringe may be used to make a skin weal with about 1% lidocaine (with or without epinephrine), and a subcutaneous channel 515 is anesthetized down to and along the course of the nerve. A scalpel is used to make an incision from about two mm to about three mm in the skin weal. The spinal needle and syringe could be used to introduce further anesthesia in the tissue along the path toward the nerve. In an embodiment, a 12-gauge 3-inch long (about 2.8 mm×about 76 mm) cannula with stylet may then be inserted to its full length through the incision and down the subcutaneous channel 515 to a position just above the nerve. In another embodiment, a 10-gauge 3-inch cannula could also be used. When placement is verified by ultrasound, the cannula stylet is removed. An implantation device 516 may be inserted to its full length through the angiocath to abut on the subcutaneous tissue. The orientation of the array 234 may be adjusted using an insertion stylet, which may be detachably attached to the array 234. Position of the implantation device 516 containing an array 234 and the nerve may then be verified with real time ultrasound. The array 234 within the implantation device may then be introduced into position over the nerve by injecting it forward through the subcutaneous channel tissue by pushing the plunger forward using a syringe-plunger implantation device 516. The position of the array 234 may be verified using in-situ stimulation. When correctly placed, the array 234 may be released from the implantation device 516. If not correctly placed, the array may be removed and repositioned.

Following successful placement, the implantation device 516 may be removed from the tissue. The distal and proximal tips of the array 234 are identified and small skin weals with lidocaine are made above them, for example using a 25-gauge ⅚-inch needle. Small (from about two mm to about three mm) incisions are made and opened by blunt dissection with a hemostat of the subcutaneous tissue until the tips of the array 234 are visible. A 5-0 absorbable suture may be attached through each end of the array through polymer suture openings or connector slots 194 at the ends of the array 234. The wounds are closed with skin glue and steristrips.

In accordance with an embodiment, the skin is prepared with antiseptic solution and draped to maintain a sterile field. Real time ultrasonography may be used in the array implant procedure to identify and locate nerves and blood vessels. Monitored anesthesia care with intravenous sedation may be used to monitor vital signs, allay anxiety, and provide pain relief. Using real time ultrasound, the nerve and blood vessel course may be outlined on the skin with an ink marker. At the proximal end of the outlined nerve, a 25-gauge 6-inch spinal needle with attached syringe may be used to make a skin weal with about 1% lidocaine (with or without epinephrine). A channel is anesthetized subcutaneously for about 4 inches from the skin weal distally along the ink outline of the nerve. An incision is made along this about 4 inch anesthetized skin area with a scalpel. Further injection of local anesthetic is used to anesthetize the subcutaneous tissues down to and along the course of the nerve. By blunt dissection with an appropriate surgical instrument, the nerve is visualized and insertion area cleared of extraneous tissues. The array implant is then placed by the physician directly over the nerve and sutures sewn through polymer eyes at the ends of the array using 5-0 absorbable material and attached to adjacent subcutaneous tissue. The wound is closed with subcutaneous sutures. The skin is closed using staples and skin glue. Ultrasound is used to verify placement.

In accordance with an embodiment, the skin is prepared with antiseptic solution and draped to maintain a sterile field. Real time ultrasonography may be used in the array implant procedure to identify nerves and blood vessels. The nerve and blood vessel course may be outlined on the skin with an ink marker. A 25-gauge 6-inch spinal needle and syringe may be used to make a skin weal with about 1% lidocaine (with or without epinephrine) and a subcutaneous channel may be anesthetized down to and along the course of the nerve. A scalpel may be used to make an about two mm to about three mm incision in the skin weal. An implantation device that includes a rectangular insertion cannula overlying an array for implantation may be inserted via the incision and down to a position above the nerve and at the desired insertion site. This may be confirmed by real time ultrasound. Using the syringe and plunger system of an implantation device, the array may be held in stable position by holding the plunger-syringe system and the rectangular insertion cannula may be removed. The array may then be released to lie over the nerve and this position may be confirmed by ultrasound. The array may be secured by placing two 5-0 sutures through the wire metal eyes at the distal and proximal end of the array 234. This may be performed via small anesthetized skin incisions placed just above each tip and identified by direct blunt dissection. The wounds are closed with skin glue and steristrips.

In accordance with an embodiment, the skin is prepared with antiseptic solution and draped to maintain a sterile field. Real time ultrasonography may be used in the array implant procedure to identify nerves and blood vessels. The nerve and blood vessel course may be outlined on the skin with an ink marker. A 25-gauge 6-inch spinal needle and syringe may be used to make a skin weal with about 1% Lidocaine (with or without epinephrine) and a subcutaneous channel may be anesthetized down to and along the course of the nerve. A scalpel may be used to make a 2-3 mm incision in the skin weal. For example, a 12-gauge 3-inch long (about 2.8 mm×about 76 mm) angiocath (or 10-gauge 3-inch is also available) may then be inserted to its full length through the incision and down the subcutaneous channel to course just above the nerve. When placement is verified by ultrasound, the catheter stylet may be removed. An implantation device may be inserted to its full length through the angiocath to abut on the subcutaneous tissue. Position of the implantation device containing the array and nerve may then be verified with real time ultrasound. The array portion of the implantation device may then be introduced into position over the nerve by injecting it forward through the subcutaneous channel tissue by pushing the plunger forward using syringe-plunger implantation device. Position of the array may be verified. If correctly placed, the array may be released from the implantation device. If not correctly placed, the array may be removed and repositioned. Following successful placement, a clip mechanism may be opened by the physician using scissor like action of the device's mechanical system and attached to the subcutaneous tissue abutting it. Alternatively, a tine on the device that may be exposed after placement may hold the device in place. The physician may close the clip on the tissue at the distal tip of the array. This action secures the array in place. The implantation device may be removed. A skin weal with local anesthetic may be raised over the proximal tip, and it may be identified by blunt tissue dissection. A 5-0 absorbable suture may be placed via the metal wire eye to the surrounding tissue. The wounds are closed with skin glue and steristrips.

In accordance with an embodiment, the implantable device 108 may be positioned on a nerve, around a nerve, or within a nerve. The electrodes 110 may be cuffed, spiked, grooved, a degradable scaffold, or any other suitable configuration.

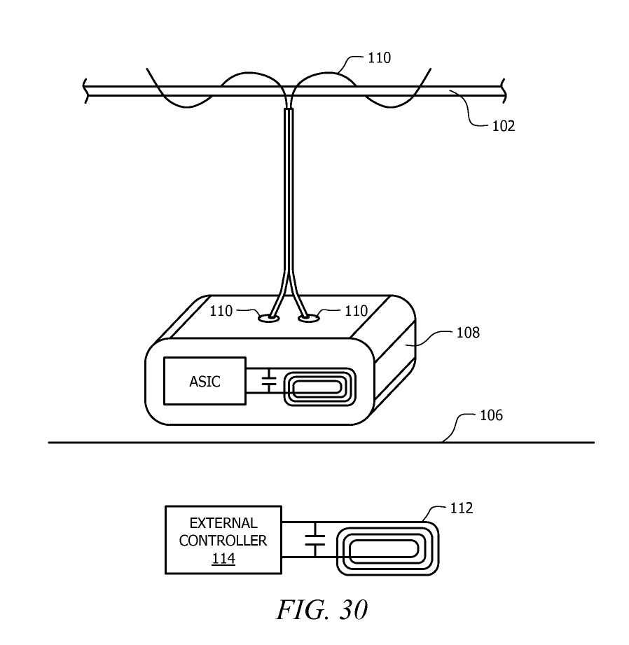

With reference to FIG. 30, an implantable device 108 is depicted with remote electrodes 110 that extend through a lead to wrap around a nerve 102, such as a vagus nerve. This embodiment permits the antenna system 112 of the implantable device 108 to be placed near the skin or tissue layer 106 while the electrodes 110 provide stimulation energy to deep nerve 102 tissue.

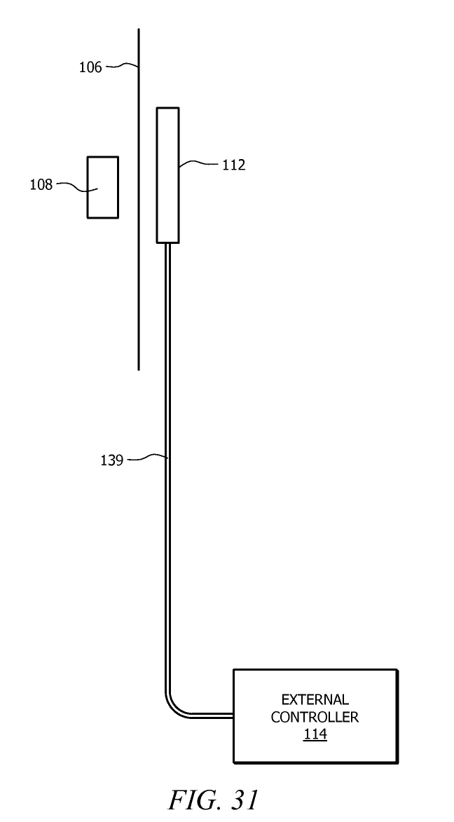

With reference to FIG. 31, an external controller 114 with remote power coils is depicted. The external controller 114 may be distanced from the external antenna or external antenna 112 with a power coil or lead 139 providing connection between them. This arrangement allows the external antenna 112 to be proximate to the implantable device 108 while the external controller 114 is maintained in a stable location. The antenna 112 and the implantable device 108 are separated by skin or tissue layer 106.

Implantable stimulators may be used to treat chronic pain by generating paresthesia in affected nerves. In particular, implantable stimulators may be used to treat carpal tunnel syndrome, diabetic foot pain, or other neuropathies, by generating paresthesia in nerves along afferent pathways. Implantable stimulators may be used to stimulate the occipital nerve, for treatment of migraines and headaches. Neural stimulation has been used to treat temporomandibular joint (TMJ), in particular with regard to associated tinnitus symptoms.

An implantable neuro stimulation array 466 may be used to provide vagus nerve stimulation. An implantable device 108 may be used to treat urinary incontinence. Neural stimulation may be used to allow therapeutics to cross the blood-brain barrier. Stimulation of certain nerves may treat conditions, particularly conditions that may be ameliorated by stimulation of a nerve. Such nerves and conditions include, but are not limited to multiple small peripheral nerves for treatment of arthritis pain, stimulation of the mandibular nerve for treatment of acute and chronic pain in the temporomandibular joint, deep brain/cortical stimulation for treatment of one or more of tremor, Parkinson's disease, dystonia, depression, tinnitus, epilepsy, stroke pain, and obsessive compulsive disorder. The nerves and conditions also include sacral nerve stimulation for the treatment of incontinence, pelvic pain and sexual dysfunction, vagus nerve stimulation for treatment of epilepsy, depression and pathoplastic conditions such as tinnitus, post traumatic stress disorder (PTSD), stroke, peripheral nerve stimulation for treatment of chronic pain, spinal cord stimulation for treatment of one or more of chronic pain, angina pain, and peripheral vascular disease pain. The nerve and conditions also include cochlear nerve stimulation for treatment of profound deafness, pulmonary nerve stimulation for treatment of respiratory support, gastric nerve stimulation for treatment of one or more of obesity, gastroparesis, and irritable bowel syndrome, and occipital nerve stimulation for treatment of headaches/migraine and/or traumatic brain injury.

The pathologies that may be treated using the disclosed systems may include one or more of the following: aberrant anatomy, acidosis, acromegalic arthritis, acromegaly, acrosclerosis, acute wasting paralysis, acute ascending paralysis, acute anterior poliomyelitis, adenohypophysis, aerobic exercise, aging, alcoholism, amyloidosis, anemia, aneurysms, angina, apoplexy, arachnodactyly, arteriolar disease, arteriosclerosis, arteriovenous malformation, artery, arthritis of rheumatic fever, arthritis, atrophic arthritis, atrophy, backward cardiac failure, baseball finger, Bence-Jones protein, benign tumor, birth palsy, blood vessel, bone marrow, bony ankylosis, brachial plexus, brachial birth palsy, brachial paralysis, brachialgia, brain, brain abscess, Buerger's disease, bulbar apoplexy, burn, bursitis, calcinosis, calcium, calcium deposits, callus, canal volume increased, canal volume decreased, capillary walls, cardiac failure, carpal bones, carpal tunnel syndrome, carpometacarpal joint arthritis, cartilage, cataract, central nervous system, cerebral vessels, cerebral palsy, cervical adenitis, cervical spine arthritis, cervical radiculopathy, Charcot's arthritis, Charcot-Marie-Tooth disorder, chorionitis, chronic arthritis, cigarette smoking, clubbed finger, collagen, coma, congestive, connective tissue, consciousness, contraceptive pills, cretinism, cyst of joint capsule, cyst of semi-lunar cartilage, cystic tumor of tendon sheath, cystic, dactylitis syphilitica, debility, deep palmar retrotlexor space abscess, deformity, degenerative joint disease, degenerative conditions, deltoid, demineralization, dermatosclerosis, diabetes, diabetes mellitus, diabetic polyneuropathy, digital neuropathies individual, digital arteries, digits, disuse osteoporosis, dolichostenomelia, dropsy, drumstick finger, dyspnea, dystrophia myotonica, ear, edema, embolism, endocrine organs, eosinophilic cells, epicondylitis, epidemic paralysis, epidermis, Erb-Duchenne syndrome, erythroid myeloma, extracranial lesions, extravasation, fascia, fibromyalgia syndrome, flaccid paralysis, fracture, ganglion, gastric, gastrointestinal, glycosuria, gonococcus, gonorrheal arthritis, gout, gouty tophus, gouty arthritis, granulation, gumma, hammer finger, Haversian spaces; head injury, heart failure, heart, Heberden's arthritis, Heine-Medin's disease, hematogenous arthritis, hematoma, hemic myeloma, hemodialysis, hemolytic streptococcus, hemophilic arthritis, hemorrhage, hereditary conditions, high blood pressure, Hippocratic finger, hunger, hydrostatic pressure, hypercalcemia, hyperemia, hyperesthesia, hyperfunction, hyperglobulinemia, hyperglycemia, hypermotility, hyperplasia, hypertension, hypertrophic pulmonary osteoarthropathy, hypertropyhic reaction, hypothyroidism, incoordination, infantile paralysis, infection, infectious arthritis, infectious conditions, inflammation, inflammatory conditions, insulin, intracranial lesions, ischemic paralysis, joint capsule, joint surface, Kahler's disease, ketosis, Klumpke palsy, knuckle, lead palsy, leprosy, lesion, leukocytosis, lipoma, lock finger, luxation, lymph, lymphocytic myeloma, lymphoid myeloma, malignant, mallet finger, malnutrition osteoporosis, Marfan's syndrome, marrow, mass lesion, median nerve entrapment, medulla oblongata, menopausal arthritis, menopause, menstruation, metabolic, migraines, mucoid hyaluronic acid, mucopolysaccharidosis, multiple sclerosis, muscle spasms, muscle tenderness, muscular dystrophy, myelocytic sarcoma, myeloid myeloma, myeloid tumor, myeloma, myeloma multiple, myofascial dysfunction, myosclerosis, myotonia dystrophica, myotonic dystrophy, myxedema, neoplastic conditions, neuritis, neurological, neurons, neuropathic arthritis, neuropathies individual, neurotrophic arthritis, obesity, obstetric paralysis, osmotic pressure, ossification, osteoarthritis, osteoporosis, palsy, paralysis, paralytic poliomyelitis, phalangectomy, phalangitis, phalangitis syphilitica, phalangization, phalangophalangeal amputation, phalanx, phosphatase, phosphorus, plasma, plasma cell myeloma, plasmacytes, plasmacytic myeloma, plasmacytic sarcoma, plasmacytoma, plasmocytic sarcoma, plasmocytic myeloma, plasmocytoma, plasmona, pneumococcus, polio, poliomyelitis, polyneuritis, pons, postinjury, postmenopausal osteoporosis, power grip, pregnancy, prenatal syphilis, printer's palsy, proliferative arthritis, pronator syndrome, protein concentration, proximal lumbrical insertion, proximal median neuropathy, psychosis of hysterical numbness and clenched-fist syndrome, pulmonary, punctiform, Quervain's disease, Quinquaud's phenomenon, radiation neuritis, radiohumeral articulation, radiohumeral bursitis, radiohumeral epicondylitio, Raynaud's syndrome, Recklinghausen's disease, reflex sympathetic dystrophy, renal failure, respiratory, reticular fibers, rheumatic fever, rheumatoid arthritis, rheumatoid tenosynovitis, ruptured disk, sanguineous apoplexy, scarlatinal synovitis, scarlatinal arthritis, scarlet fever, gcleriasis, scleroderma, seal finger, seizures idiopathic peripheral causes, senile, osteoporobis, senility, skeleton, sodium urate, spastic diplegia, spider finger, spinal nerves, spinal cord, spinal cord lesions, splanchnomegaly, sprain fracture, springfinger, Steinert's disease, stellate cells, stroke, subchondral spaces,' subdural hematoma, sugar tolerance, swallow, swelling, symmetrical synovitis, syndactyly, synovectomy, synovial, synovial tissues, synovial distention, synovial proliferation nonspecific, synovitis, synovium nonspecific fibrosis, syphilitic arthritis, syringomyelia, systemic disease, tabes dorsalis, tactile anesthesia, tendinitis, tendon sheath, tendons, tendovaginitis stenosans, tenosynovitis, thirst, thoracic outlet syndrome, thrombosed, thrombosis, thyroid hormone, thyroid conditions, tophaceous gout, trabeculae, transient ischemic attacks, trauma, traumatic conditions, tremors, trigger finger, tuberculosis, tuberculous tenosynovitis, tuberous sclerosis, tumor, tumor benign, ulnar nerve neuropathy, uremia, uric acid, urine, vascular spasm, vascular conditions, vasculitis, vasomotor disturbances, viscera, volkmanrla paralysis, weakness, webbed fingers, weeping sinew, weight, white-finger syndrome, or wrist malalignment.

None of the description in the present application should be read as implying that any particular element, step, or function is an essential element that must be included in the claim scope: the scope of patented subject matter is defined only by the allowed claims. Moreover, none of these claims is intended to invoke paragraph six of 35 USC section 112 unless the exact words “means for” are followed by a participle. The claims as filed are intended to be as comprehensive as possible, and no subject matter is intentionally relinquished, dedicated, or abandoned.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 5, 4, etc.; greater than 0.10 includes 0.11, 0.12, 0.15, etc.). For example, whenever a numerical range with a lower limit, Rₗ, and an upper limit, Rᵤ, is disclosed, any number falling within the range is specifically disclosed. In particular, the following numbers within the range are specifically disclosed: R=R₁+k*(Rᵤ−Rₗ), wherein k is a variable ranging from 1 percent to 100 percent with a 1 percent increment, i.e., k is 1 percent, 2 percent, 5 percent, 4 percent, 5 percent, . . . , 50 percent, 51 percent, 52 percent, . . . , 75 percent, 76 percent, 77 percent, 78 percent, 77 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure. The discussion of a reference in the disclosure is not an admission that it is prior art, especially any reference that has a publication date after the priority date of this application. The disclosure of all patents, patent applications, and publications cited in the disclosure are hereby incorporated by reference, to the extent that they provide exemplary, procedural, or other details supplementary to the disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

What is claimed is:

1. A neuro stimulation array comprising:
a first implantable neurostimulator storing a first identification code in a non-volatile memory and responding to communications including said first identification code;
a second implantable neuro stimulator storing a second identification code in a non-volatile memory and responding to communications including said second identification code; and
a polymer connector attached to said first implantable neurostimulator and said second implantable neuro stimulator, thereby forming a neuro stimulation array.

2. The neurostimulation array of claim 1, further comprising an end piece attached to said first implantable neurostimulator, said end piece including a suture hole.

3. The neuro stimulation array of claim 1, wherein said first implantable neuro stimulator stores a first local identification code in volatile memory and responds to communications including said first local identification code, and wherein said second implantable neurostimulator stores a second local identification code in volatile memory and responds to communications including said second local identification code.

4. The neurostimulation array of claim 1, further comprising a third implantable neurostimulator attached between said first implantable neurostimulator and said second implantable neuro stimulator.

5. The neurostimulation array of claim 1, wherein a polymer layer covers said first implantable neurostimulator and said second implantable neurostimulator.

6. The neurostimulation array of claim 5, wherein electrodes on a surface of the first implantable neurostimulator are exposed by holes in said polymer layer.

7. The neurostimulation array of claim 1, wherein the first and second implantable neurostimulators each include a battery.

8. The neurostimulation array of claim 1, wherein the first and second implantable neurostimulators do not include a battery.

9. The neurostimulation array of claim 1, wherein the first and second implantable neurostimulators are each powered inductively.

10. An implantable neurostimulation array system comprising:
an array of neurostimulators, wherein each neurostimulator is represented by an individual identification code; and
an external controller in communication with the neurostimulators,
wherein a communication from the external controller to one of the neurostimulators comprises the individual identification code representing the one of the neurostimulators,
wherein the external controller supplies power modulated with the communication to the array of neurostimulators, and
wherein the neurostimulators communicate with the external controller by modulating an antenna load.

11. The implantable neurostimulation array system of claim 10, wherein a connecting material is positioned between two of the neurostimulators.

12. The implantable neurostimulation array system of claim 10, wherein a connecting material covers two of the neurostimulators.

13. The implantable neurostimulation array system of claim 10, wherein electrodes on a surface of the neurostimulators are exposed by holes in the connecting material.

14. The implantable neurostimulation array system of claim 10, wherein each of the neurostimulators comprises a battery.

15. The implantable neurostimulation array system of claim 10, wherein each of the neurostimulators is powered inductively.

16. The implantable neurostimulation array system of claim 10, wherein an end piece is coupled to one of the neurostimulators and provides a suture hole connector for the one of the neuro stimulators.

17. The implantable neurostimulation array system of claim 10, wherein the neurostimulators provide voltage controlled capacitive discharge stimulations.

18. An implantable voltage controlled capacitive discharge neurostimulation system comprising:
an external controller; and
an implantable neurostimulator in communication with the external controller,
wherein the implantable neurostimulator provides voltage controlled capacitive discharge stimulation energy.

19. The implantable voltage controlled capacitive discharge neurostimulation system of claim 18, wherein the implantable neurostimulator comprises a battery.

20. The implantable voltage controlled capacitive discharge neurostimulation system of claim 19, wherein the implantable neurostimulator is powered inductively.

21. An implantable voltage controlled capacitive discharge neurostimulator comprising:
a power source; and
a plurality of electrodes coupled to the power source,
wherein the power source provides voltage controlled capacitive discharge stimulation energy to the electrodes.

22. The implantable voltage controlled capacitive discharge neurostimulator of claim 21, wherein the power source is a battery.

23. The implantable voltage controlled capacitive discharge neurostimulator of claim 21, wherein the power source is an inductive power source.

24. A neurostimulation device comprising:
a housing that has a substantially rectangular shape including a first side and a second side on opposite sides of the substantially rectangular shape;
a first electrode located on the first side; and
a second electrode located on the second side.

25. The neurostimulation device of claim 24, wherein a plurality of first electrodes are located on the first side and a plurality of second electrodes are located on the second side.

26. The neurostimulation device of claim 25, wherein the first electrodes comprise a plurality of tri-state electrodes and the second electrodes comprise a plurality of tri-state electrodes.

27. A neurostimulation system comprising:
an implantable neurostimulation device identified by an identification code; and
an external device in communication with the implantable neurostimulation device,
wherein the external device receives the identification code from the implantable neurostimulation device, assigns a local identification code to the implantable neurostimulation device for use in future communication, and transmits the local identification code to the implantable neurostimulation device; and
wherein the local identification code is stored in memory in the implantable neurostimulation device.

28. The neurostimulation system of claim 27, wherein the identification code is more than 20 bits.

29. The neurostimulation system of claim 27, wherein the local identification code is less than about 20 bits.

30. A neurostimulation system comprising:
an implantable neurostimulation device comprising a housing; and
an imaging system for detecting the implantable neurostimulation device,
wherein the housing is substantially radio-opaque to the imaging system from a first direction and substantially radio-transparent to said imaging system from a second direction.

31. The neurostimulation system of claim 30, wherein the first direction is perpendicular to the second direction.

32. A neurostimulation system comprising:
an implantable neurostimulation device including an internal inductive coil; and
an external control device configured to communicate inductively with the implantable neurostimulation device,
wherein the implantable neurostimulation device communicates with the external control device by changing the inductance of the internal inductive coil.

33. The neurostimulation system of claim 32, wherein said change of inductance is caused by shorting the internal inductive coil.

34. The neurostimulation system of claim 32, wherein said external control device provides power to the implantable neurostimulation device inductively.

35. A neurostimulation device comprising:
a housing comprising a metallic electrode, wherein the housing does not cover a portion of the metallic electrode; and
a non-conductive coating that covers at least part of the portion of the metallic electrode.

36. The neurostimulation device of claim 35 further comprising a stimulation circuit coupled to the metallic electrode and that provides voltage controlled capacitive discharge stimulation energy to the metallic electrode.

37. A neurostimulation device comprising:
a rectangular shaped housing that has a first side and a second side;
a first electrode on the first side; and
a second electrode on the second side.

38. The neurostimulation device of claim 37, wherein said first side is opposite to said second side.

39. The neurostimulation device of claim 37, wherein said first side is parallel to said second side.

40. A neurostimulation device comprising:
a housing having a first face and a second face;
a plurality of tri-state electrodes on the first face; and
a plurality of tri-state electrodes on the second face.

41. The neurostimulation device of claim 40, wherein said first side is opposite to said second side.

42. The neurostimulation device of claim 40, wherein said first side is orthogonal to said second side.

43. The neurostimulation device of claim 40, wherein said tri-state electrode can be positive, negative or off.

44. An implantable device with a hermetic seal comprising:
a first alumina shell piece having a first bonding surface covered with a gold-tin epoxy;
a second alumina shell piece having a second bonding surface covered with a gold metallization;
wherein the first alumina shell piece is bonded to the second alumina shell piece such that the first bonding surface is aligned with the second bonding surface and the gold-tin epoxy and the gold metallization form a hermetic seal.

45. The implantable device of claim 44, further comprising a bio-compatible material covering said hermetic seal.
